(19) United States
(12) Patent Application Publication
(10) Pub. No.: US 2023/0070045 A1
(43) Pub. Date: Mar. 9, 2023

(54) AN INJECTION DEVICE WITH INTEGRATED NEEDLES

(71) Applicant: Novo Nordisk A/S, Bagsvaerd (DK)

(72) Inventors: Henrik Bengtsson, Taastrup (DK); Ronan James Carroll, Daleen (IE); Bo Kvolsbjerg, Helsingoer (DK)

(21) Appl. No.: 17/798,959

(22) PCT Filed: Feb. 16, 2021
(86) PCT No.: PCT/EP2021/053752

§ 371 Date: Aug. 11, 2022

(30) Foreign Application Priority Data
Feb. 18, 2020 (EP) 20157959.6

(51) Int. Cl. A61M 5/315; A61M 5/20

(52) U.S. Cl. CPC A61M 5/31595; A61M 5/20; A61M 2005/004

(57) ABSTRACT

The invention relates to an injection device for ejecting a predetermined plurality of fixed doses. The doses are expelled by moving a needle shield (30) in the proximal direction which releases a pre-strained torsion spring (115) to eject one of the predetermined doses at the time. The injection device is further provided with a number of integrated needle assemblies (70) which one at the time are brought to an injection position. The needle change mechanism (80,90) operating the needle assemblies is controlled by rotation of the needle shield which is rotatable between a locked and an unlocked position.

### THE TECHNICAL FIELD OF THE INVENTION

The present invention relates to an injection device for ejecting a predetermined plurality of fixed doses. Further, the present invention relates to such injection device having a plurality of integrated needle assemblies wherein each of the needle cannula assemblies can be brought to an injection position for ejecting one of the fixed doses.

The present invention thus relates to a so-called Multi-Use Fixed Dose Device which is able to deliver a predefined plurality (i.e. more than one) of fixed doses of a liquid drug to a user.

In a preferred example the number of fixed doses and the number of needle assemblies are the same.

### DESCRIPTION OF RELATED ART

Examples of injection devices provided with an integrated needle cannulae are disclosed WO 2001/93927 and in US 2012/0016315.

WO 2001/93927 discloses a needle magazine which in one example is formed as an integral part of an injection device. During use, the user manually pushes a shield axially. The manual axial movement of the shield in the proximal direction brings a needle cannula into a relative injection position wherein the entire front-end of the needle cannula is exposed outside the shield and the back-end of the needle cannula is penetrated into the cartridge which is secured in the injection device. With the needle cannula positioned in this injection position, the user can expel any number of doses wanted through the same needle cannula. The needle shield is preferably moved proximally by a spring when the user decides to manually release the shield. When the shield has been moved to its proximal position, the user can manually rotate the shield to thereby position the shield in the correct rotatable position for the next needle cannula. There is no automatic correlation between the release mechanism and the needle change mechanism as each mechanism is individually operated by the user.

US 2012/0016315 discloses an injection device with a needle magazine which in one example can form one unitary disposable assembly. The individual needle cannulae are placed in a hinged structure such that one individual needle cannula at the time can be brought into contact with the cartridge.

Common for such integrated solutions is that the injection device with the build-in needle magazine must be discarded either when all the needle cannulae are used or when the volume of liquid drug contained in the injection device is used. Depending on what occurs first, the user is forced to either discard unused needle cannulae or unused liquid drug.

Further, both the needle change, and the release of the dose are done as two individual user operations.

As an alternative to injection devices with integrated needle magazine are stand-alone needle magazines which the user manually attaches to an injection device. Examples of such separate needle magazines are disclosed in WO 2017/189164 and in U.S. Pat. No. 9,889,249.

After having attached such needle magazine to an injection device, the user must manually operate the needle magazine to bring the individual needle cannulae into an injection position. Since the needle magazine is a stand-alone unit there is no correlation between the needle change and the ability to inject a dose as these two actions are made separately.

Another drawback with such solution is that the user is faced with several kinds of handling issues. Firstly, it is not recommendable to move the needle magazine to a new injection device in case the injection device runs dry from liquid drug before all the individual needle cannulae are used since the sterility barrier protecting the back-end needle is broken first time the needle magazine is attached to an injection device. Moving the needle magazine from one injection device to another one would seriously expose the back-end of the needle cannulae entering the cartridge to contamination and in order to avoid this, the user is forced to discard the needle magazine when the injection device runs dry for liquid drug and hence discard unused needle cannulae.

Secondly, if all the needle cannulae in the attached needle magazine are used while there is still liquid drug left in the injection device, the user must replace the needle magazine with a new needle magazine where after the possibility of running dry for liquid drug in the injection device before all the needle cannulae in the new magazine are used is fairly high which would again lead to the user discarding unused needle cannulae.

A further obstacle with both the integrated solutions and the separate needle magazines are the fact that the changing of the individual needle cannulae is performed manually which means that the same needle cannula can accidentally be used for several injections should the user forget to change to a new needle cannula.

### DESCRIPTION OF THE INVENTION

It is an object of the present invention to provide an injection device with integrated needle cannulae wherein the safety for the user has been optimized and wherein it is ensured that each dose is delivered using an unused needle cannula.

Accordingly, in a first aspect of the invention a pre-filled injection device for releasing a pre-defined plurality of fixed doses of a liquid drug is provided. The injection device comprises:
A housing structure which is provided with a container containing the liquid drug to be released. The container, which in a preferred example is a cartridge, contains the predetermined number of substantially equally sized dose volumes.
A release mechanism for releasing the predefined plurality of fixed doses one at the time as a first dose followed by a number of next doses. The fixed doses preferably has substantially the same volume.
A plurality of needle assemblies which are integrated in the housing structure and individually movable into an injection position.
A needle change mechanism which when activated preferably by a user, moves one needle assembly into the injection position, and wherein
   the release mechanism and the needle change mechanism cooperate such that each of the next doses can only be released after at least one activation of the needle change mechanism.

The user consequently has to activate the needle change mechanism and move a new needle assembly into the injection position before it is possible to release the next subsequent fixed dose.

The release of each dose can be foreseen in many different ways e.g. by activating a release button or by a spring driven mechanism which in one example could be triggered by the movement of a further mechanism such as a needle shield.

However, the release of each of the next doses following the first dose requires that the needle change mechanism has been operated at least once such that a new and unused needle assembly has been moved to the injection position prior to ejecting each of the next doses.

In this way it can be ensured that each of the doses in the injection device is ejected through an unused needle assembly.

A fixed dose is generally a dose having a fixed volume which is determined by the manufacturer of the injection device. When such injection device is capable of ejecting a plurality of fixed doses, the doses in the plurality can have minor variations due to mechanical tolerances in the injection device. However, all the fixed doses delivered by the injection device preferably has substantially the same volume and the term “fixed doses” are meant to cover a predetermined and fixed number of substantially equally sized dose volumes.

Further, it is preferred that the number of needle assemblies available in the plurality of needle assemblies is the same as, or more than, the predefined number of fixed doses of liquid drug available in the predetermined plurality of fixed doses. The last available dose is thus always injected with a new needle assembly where after the user discards the injection device.

Preferably, the first dose is injected using the first needle assembly where after the user activates the needle change mechanism thus bringing a new needle assembly into the injection position such that the second dose is injected by the second needle assembly and so forth. Each fixed dose in the predetermined plurality of doses are thus injected through a new needle assembly and the pre-filled injection device runs out of fixed doses before it runs out of needle assemblies.

In one example, four fixed doses are available and four (or more) needle assemblies are available. Hence each fixed dose can be injecting through a new and clean needle assembly.

In one example a needle shield can be provided which needle shield is telescopically movable in relation to the housing structure between a first position to a second position.

The first position of the needle shield is preferably a position wherein the needle shield is extended in an axial direction to cover the needle cannula. To cover is here meant that the needle shield extends over and covers the needle cannula in the axial direction, not necessarily in the radial direction.

The second position of the needle shield is preferably a position wherein the needle shield has been moved proximally to thereby expose at least the distal end of the needle cannula.

The release of each of the doses in the predefined plurality is preferably released when moving the needle shield axially from the first position and into the second position thus making the injection device a so-called shield triggered device i.e. an injection device wherein the axial movement of the needle shield activates the release of the dose.

In one example, the needle shield is guided translational in its movement from the first position to the second position.

In a further example, the release mechanism comprises a connector element and a torsion spring driven drive tube for driving a piston rod.

The connector thus connects the needle shield with the drive tube and the drive tube is further connected to the housing structure by a torsion spring. The drive tube is thus pushed proximally out of engagement with the housing structure by the connector which downstream is operated by the needle shield. Each time the drive tube is pushed out of engagement with the housing structure the drive tube is rotated by the torsion spring. In one preferred example, the drive tube rotates one full revolution each time it is pushed out of its engagement. The rotation of the drive tube generates a distal movement of the piston rod such that the fixed dose is expelled.

The dose volume being released is thus a result of the rotation of the drive tube and the transformation of this rotation to a movement of the piston rod in the distal direction. Since these interfaces are the same for all doses and the drive tube is moved the same distance for each release, the same dose volume will be expelled each time the drive tube is moved out of engagement with the housing structure. However, minor tolerances can occur.

The needle shield is in one example rotatable between a locked and an unlocked position when the needle shield is in the first position.

The user is thus able to lock and unlock the injection device by rotation of the needle shield once the needle shield is in its extended first position.

In the locked position it is not possible to activate the release mechanism since the needle shield in the locked position is hindered in any axial movement.

In a further example, the protective cap is used to rotate the needle shield between the locked and the unlocked position. This is preferably done by having the protective cap engage with the housing structure via a track interface that requires the user to rotate the protective cap in order to remove it (and attach it) and to transform this rotation of the protective cap to a rotation of the needle shield

Each needle assembly preferably comprising a hub adapted to hold a needle cannula such that a distal end points in a distal direction and a proximal end points in a proximal direction.

Further, in one example the container containing the liquid drug is a cartridge having an interior containing the liquid drug. The interior is defined by a movable plunger and a pierceable septum. In such construction which is well known from many different injection devices, the needle cannula used for injecting is pierced through the septum and the plunger is moved axially to thereby decrease the volume of the interior such that a quantum of the liquid drug is forced out through the lumen of the needle cannula.

When using a cartridge as the container, the cartridge is preferably permanently secured in the housing structure thereby making the injection device a pre-filled injection device.

Usually a drive mechanism is present for driving the plunger forward inside the cartridge. In the present example, the drive mechanism is a release mechanism which releases one fixed dose at the time.

First time the needle shield is rotated to unlock the injection device, all the proximal ends of all the needle cannulae in the plurality of needle assemblies are pierced through the septum of the container upon the first rotation of the needle shield from the locked position to the unlocked position.

Once the proximal ends of all the needle cannulae is inserted into the container, the proximal ends remain inserted into the container for the remaining life-time of the injection device. Whenever a needle cannula is moved distally to the injection position, the proximal end of that particular needle cannula remains inserted into the interior of the container but is translated a distance in the distal direction thus moving the proximal end of the needle cannula closer to the septum of the cartridge. Following an injection, the needle cannula is moved proximally such the proximal end of that particular needle cannula is moved back to its initial position. The distance the distal ends of the needle cannulae are moved when moved from the storage position and into the injection position must thus be shorter or at least the same as the distance the proximal ends are inserted into the cartridge the first time the user rotates the needle shield in order to maintain the proximal ends inserted into the container.

In a preferred example, both the release mechanism and the needle change mechanism are activated by movement of the needle shield.

The needle shield thus has two patterns of movements. During injection, the needle shield is translated forth and back and during locking and unlocking of the injection device, the needle shield is rotated relatively to the housing structure.

These two patterns of movements (translation and rotation) are used to both activate the release of a dose and to change the needle assembly.

In one example, a new and unused needle assembly is moved to the injection position upon each rotation of the needle shield from the locked position to the unlocked position.

The needle change mechanism is preferably such that whenever the user rotates the needle shield to the locked position, an old and used needle assembly is moved proximally into the storage position and whenever the user rotates the needle shield to the unlocked position in preparation of a new dose release, the next new and unused needle assembly is moved into the injection position.

The needle change mechanism preferably comprises a key and a carousel which both have a helical track used to move the needle assembly axially.

Translation of the needle shield forth and back as done during injection, generates a rotation the key such that the key is moved to the next unused needle assembly during injection.

Following an injection, once the needle shield is back in the first position, the user rotates the needle shield to the locked position. This rotation of the needle shield generates a simultaneously rotation of the carousel and the helical track inside the carousel moves the needle assembly.

The implications are that whenever the needle shield is rotated to the locked position, the old and used needle assembly is moved proximally and whenever the key and the carousel are rotated in the same rotational direction a new and unused needle assembly is moved into the injection position.

During rotation of the needle shield to the locked position, the key needs to be maintained in the new position it has been moved to during injection. It is thus desirable to have click means which rotates the key together with the needle shield and the carousel when moving a new needle assembly to the injection position but which maintains the key in its position whenever the needle shield and the carousel are rotated to the locked position and the old and used needle assembly is moved back to the storage position.

### DEFINITIONS

An “injection pen” is typically an injection apparatus having an oblong or elongated shape somewhat like a pen for writing. Although such pens usually have a tubular cross-section, they could easily have a different cross-section such as triangular, rectangular or square or any variation around these or other geometries.

The term “Needle Cannula” is used to describe the actual conduit performing the penetration of the skin during injection. A needle cannula is usually made from a metallic material such as e.g. stainless steel and preferably connected to a hub made from a suitable material e.g. a polymer. A needle cannula could however also be made from a polymeric material or a glass material.

As used herein, the term “Liquid drug” is meant to encompass any drug-containing flowable medicine capable of being passed through a delivery means such as a hollow needle cannula in a controlled manner, such as a liquid, solution, gel or fine suspension. Representative drugs could include pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

“Cartridge” is the term used to describe the primary container actually containing the liquid drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end of the cartridge is typically closed by a plunger or piston made from a rubber composition or a suitable polymer. The plunger or piston can be slidable moved inside the cartridge. The space between the pierceable membrane and the movable plunger holds the liquid drug which is pressed out as the plunger decreased the volume of the space holding the liquid drug.

The cartridges used for both pre-filled injection devices and for durable injections devices are typically factory filled by the manufacturer with a predetermined volume of the liquid drug. A majority of the cartridges currently available contains either 1.5 ml or 3 ml of liquid drug.

Since a cartridge usually has a narrow distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

By the term “Pre-filled” injection device is meant an injection device in which the cartridge containing the liquid drug is permanently embedded in the injection device such that it cannot be removed without permanent destruction of the injection device. Once the predetermined amount of liquid drug in the cartridge is used, the user normally discards the entire injection device. Usually the cartridge which has been filled by the manufacturer with a specific amount of liquid drug is secured in a cartridge holder which is then permanently connected in a housing structure such that the cartridge cannot be exchanged.

This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

A “Multi-Use Fixed Dose” injection device is meant to define an injection device which is able to deliver a predefined plurality (i.e. more than one) of doses which are substantially identical in volume. The liquid drug contained in the cartridge is thus expelled in a number of substantially identical dose volumes. In one example the cartridge could e.g. contain 3 ml of liquid drug which could e.g. be expelled in 6 identical doses each of 0.5 ml. The number of equally sized doses are often 2 to 8, and preferably 4 to 6 identical dose volumes. A multi-use fixed dose injection device can either be pre-filled such that the injection device is discarded after the predefined number of dose volumes has been expelled or it can be a durable injection device enabling the user to change the cartridge and expel a new series of equally sized doses volumes from the new cartridge.

Using the term “Automatic” in conjunction with injection device means that, the injection device is able to perform the injection without the user of the injection device delivering the force needed to expel the liquid drug during dosing. The force is typically delivered—automatically—by an electric motor or by a spring drive. The actual spring for the spring drive is e.g. strained by the user during dose setting, however, such springs are usually pre-strained with a low force in order to avoid problems of delivering very small doses. Alternatively, the spring can be fully preloaded by the manufacturer with a preloaded force sufficient to expel the full initial content (i.e. the entire injectable content) of liquid drug contained in the cartridge though a number of doses. Typically, the user activates a latch mechanism provided either on the surface of the housing or at the proximal end of the injection device to release—fully or partially—the force accumulated in the spring when carrying out the injection.

The term “Permanently connected” or “permanently embedded” as used in this description is intended to mean that the parts, which in this application is embodied as a cartridge permanently embedded in the housing structure, requires the use of tools in order to be separated and should the parts be separated it would permanently damage at least one of the parts thus rendering the injection device unable to operate.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 shows perspective views of the injection device

FIG. 2 shows an exploded view of the distal part of the injection device.

FIG. 3 shows different views of the shield guide.

FIG. 4 shows different views of the protective cap.

FIG. 5 shows different views of the Needle shield.

FIG. 6 shows different views of the tower.

FIG. 7 shows different views of the tip part.

FIG. 8 shows different vies of the needle assemblies.

FIG. 9 shows different views of the key.

FIG. 10 shows different views of the carousel.

FIG. 11 shows an exploded view of the release mechanism

FIG. 12A-L shows the different states of the injection device.

FIG. 12M shows the injection device ready to expel a next dose.

FIG. 13A-D shows the distal end of the injection device with the needle unit.

FIG. 14A-B shows the injection device unlocked and ready to inject the first dose.

FIG. 15A-B shows the injection device with the needle shield sliding proximally.

FIG. 16A-B shows the injection device during injection of the first dose.

FIG. 17A-B shows the injection device with the needle shield sliding distally.

FIG. 18A-B shows the injection device with the needle shield fully removed from the skin.

FIG. 19A-B shows the injection device as the needle shield is rotated into the locked position.

FIG. 20A-B show the injection device with the needle shield in the locked position.

FIG. 21A-B shows the injection device with the needle shield being rotated into the unlocked position.

FIG. 22 shows the injection device ready to expel a next dose.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clock-wise” and “counter clock-wise” or similar relative expressions are used, these only refer to the appended figures and not to an actual situation of use. The shown figures are schematic representations for which reason the configuration of the different structures as well as their relative dimensions are intended to serve illustrative purposes only.

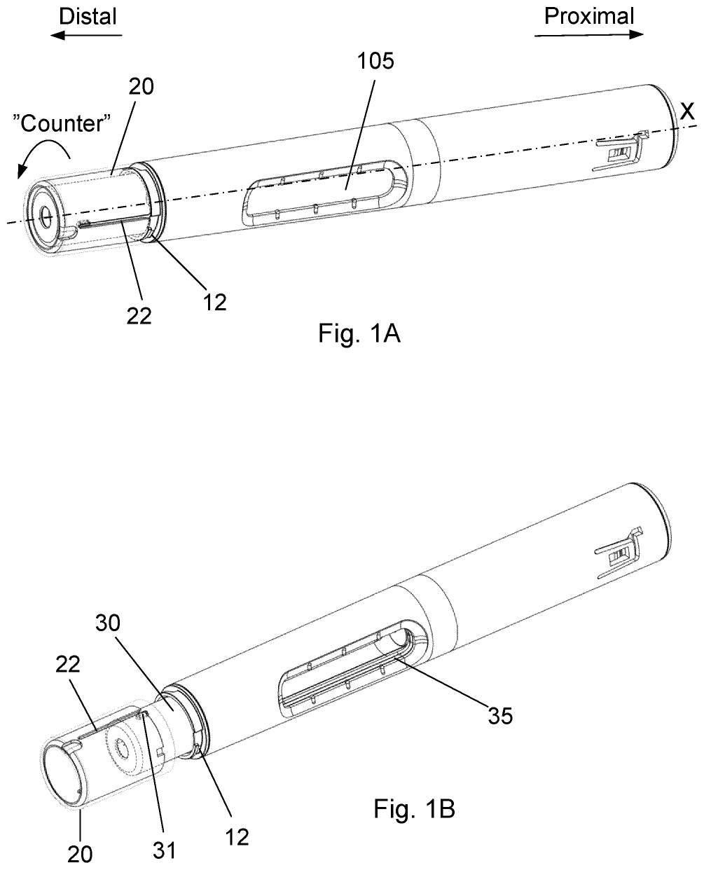

In that context it may be convenient to define that the term “distal end” in the appended figures is meant to refer to the end of the injection device carrying the needle cannulae and pointing towards the user during injection, whereas the term “proximal end” is meant to refer to be the opposite end as indicated in FIG. 1A. Distal and proximal are meant to be along an axial orientation extending along the longitudinal centre axis (X) of the injection device as also disclosed in FIG. 1A.

When referring to clock-wise and counter clock-wise in the following examples it is understood that the injection device is viewed from a position distal to the injection device. Clockwise is thus rotation following the direction of the arms on a clock and counter clock-wise is towards the opposite rotational direction as indicated by the arrow “Counter” in FIG. 1 (which depicts the counter clock-wise direction).

To explain the various movements taken place in the injection device described, the following terminology are used throughout the following examples;

“Translational movement” is meant to be a strictly linear movement without any rotation.

“Rotational movement” is any movement of rotation around a centre which centre can be a centre point i.e. in one planar or a centre axis i.e. having a longitudinal extension.

“Axial movement” means any movement in an axial direction e.g. along the centre axis. Such movement can be a strictly translational movement or include a rotational movement which thus makes it a “Helically movement” as this is meant to be the combination of an axial movement and a rotational movement.

“Telescopic” is meant to cover the situation in which a movable element moves out from, or into, a base element. The movement can be translational i.e. without any rotation, or the movement can include a rotation thus making the movement helical.

In the following example reference is made to the dose expelling mechanism of the multi-use fixed dose injection device disclosed in International patent application No. PCT/EP2000/085271 which is henceforth included by reference.

In the example herein provided, the multi-use fixed dose device is designed to expel four (4) equally sized (i.e. fixed) dose volumes. However, any other random number of fixed doses can be envisaged, e.g. 2, 3, 5, 6 or more fixed dose volumes.

FIG. 1A-B discloses the injection device in a perspective view. The housing structure is provided with a window 105 which has markers indicating the number of fixed doses and distally the needle carrying end of the injection device is covered by a removable protective cap 20 which the user can remove as shown in FIG. 1B.

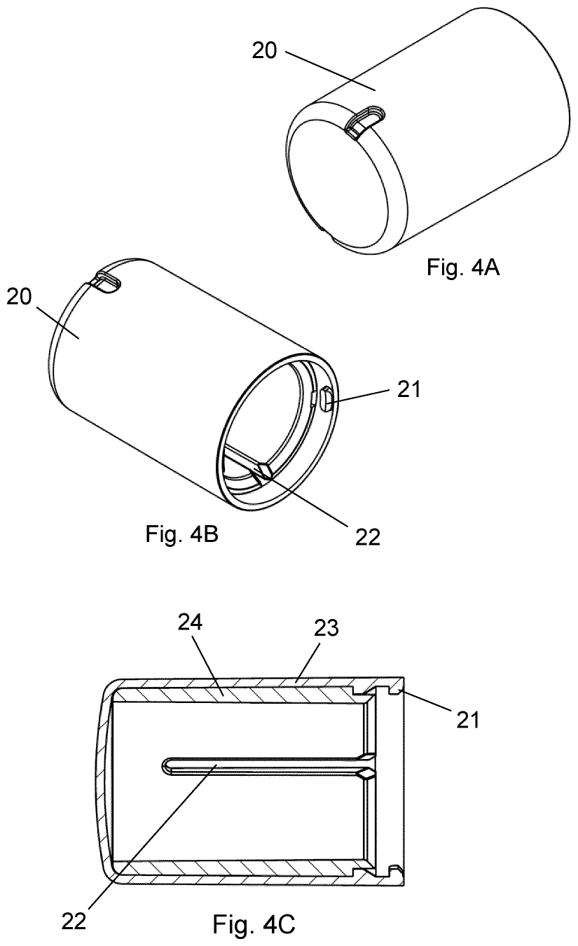

The protective cap 20 which is disclosed in further details in FIG. 4A-C has an outer shell 23 and an inner shell 24 and is preferably moulded in a 2K moulding. The inner shell 24 has a longitudinal track 22 and the outer shell 23 is provided with one or more inner protrusions 21. To better visualize the longitudinal track 22, the outer shell 23 is shown with broken lines in FIG. 1A-B.

As seen in FIG. 1A-B wherein the first shield 30 is shown with broken lines when lying under the protective cap 20, the inner protrusions 21 engages in a peripheral track 12 provided in the shield guide 10. The user is henceforth forced to rotate the protective cap 20 before it can be removed. During the counter clock-wise rotation of the protective cap 20 from FIG. 1A to FIG. 1B, the longitudinal track 22 rotates the first shield 30 via the engagement with the outwardly pointing bars 31 on the first shield 30.

The needle shield 30, 35 which comprises the first shield 30 and the second shield 35 as will be explained is also provided with a window which can be rotated into alignment with the window 105 in the housing structure thus allowing the user to visually inspect the content of the cartridge before performing an injection.

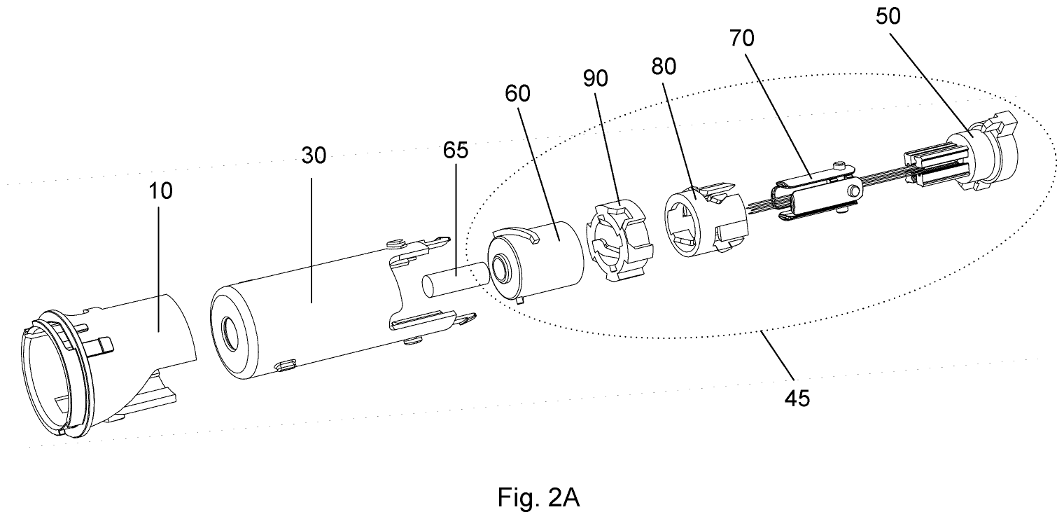

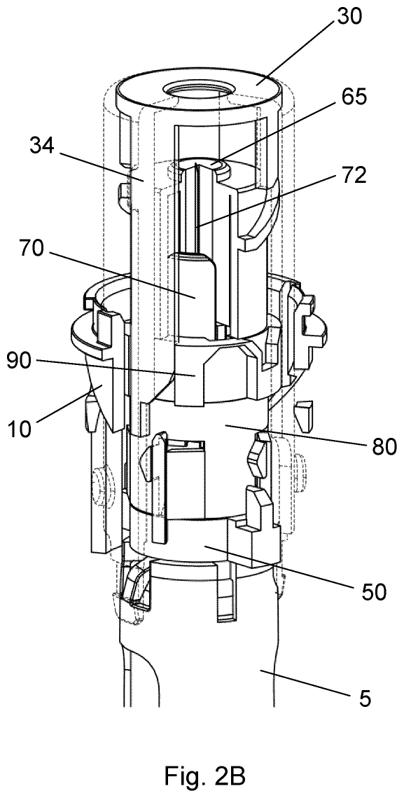

FIG. 2A-B disclose the distal part of the injection device and comprises when seen from the distal end of the injection device (in FIG. 2A):
a shield guide 10 which is a part of the housing structure,
a telescopically movable first shield 30, and
a needle unit which is encircled in FIG. 2 and referenced 45.

In FIG. 2B the distal part of the injection device has been visually cut open to visualize the interior of the needle unit 45 and the outer contour of the needle shield is shown in broken lines. The needle unit 45 is in a unitary part formed from a tower 50 and a tip part 60. The tower 50 and the tip part 60 are permanently connected e.g. by gluing, welding or by a click-fit and the axial void 62 in the tip part 60 is sealed by a pierceable plug 65 which is press fitted into the axial void 62.

The pierceable plug 65 is made from a suitable polymer which in one example could be the same TPE as used for the septum 8 in the cartridge 6. Preferably, the pierceable plug 65 also contains an anti-bacterial additive.

The needle unit 45 holds the individual needle assemblies 70 together with a needle change mechanism which comprises a key 80 and a carousel 90. The needle change mechanism is driven by the needle shield 30 as will be explained.

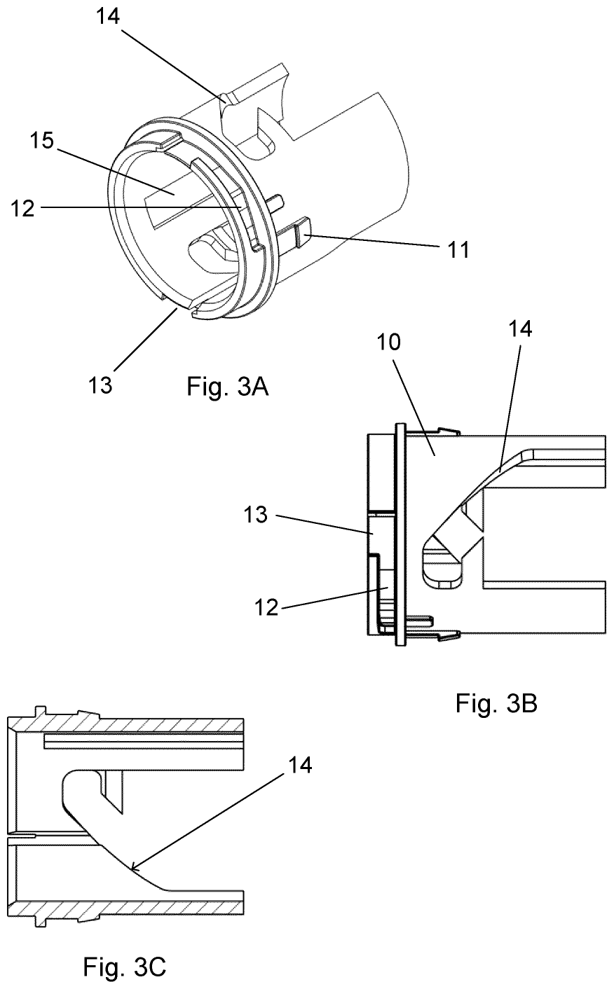

The above individual parts are disclosed in further details in the FIGS. 3 to 10.

FIG. 3A-C shows the shield guide 10 which is part of the overall housing structure which forms the outer boundaries of the injection device. The shield guide 10 is provided with a number of click arms 11 by which the shield guide 10 can be clicked into the housing part 100 such that the shield guide 10 and the housing part 100 becomes one united housing structure. Preferably, two such click arms 11 are provided. Alternatively, the shield guide 10 can be moulded as an integral part of the housing structure.

The shield guide 10 is distally provided with a peripheral track 12 which guides the inner protrusion 21 provided inside the protective cap 20. This peripheral track 12 has at least one axial track opening 13. The user is henceforth required to rotate the protective cap 20 before the inner protrusion 21 inside the protective cap 20 is aligned with the axial track opening 13 and the protective cap 20 can be axially pulled off the housing structure.

The bottom of the peripheral track 12 preferably has a raised part such that the inner protrusion 21 inside the protective cap 20 encounters some resistance when the protective cap 20 is rotated relatively to the shield guide 10 and thus the housing structure. This provides the user with a tactile indication when the protective cap 20 is removed and attached.

The protective cap 20 which protects the distal part of the injection device between injections is disclosed in FIG. 4A-C and comprises the inner protrusion 21 engaging the peripheral track 12. Any number of protrusions 21 can be provided, however two are preferred such that the protective cap 20 needs to be rotated less than 180° before it can be pulled axially off. The inner shell 24 of the protective cap 20 is further provided with a longitudinal track 22. One or more of such longitudinal tracks 22 can be provided.

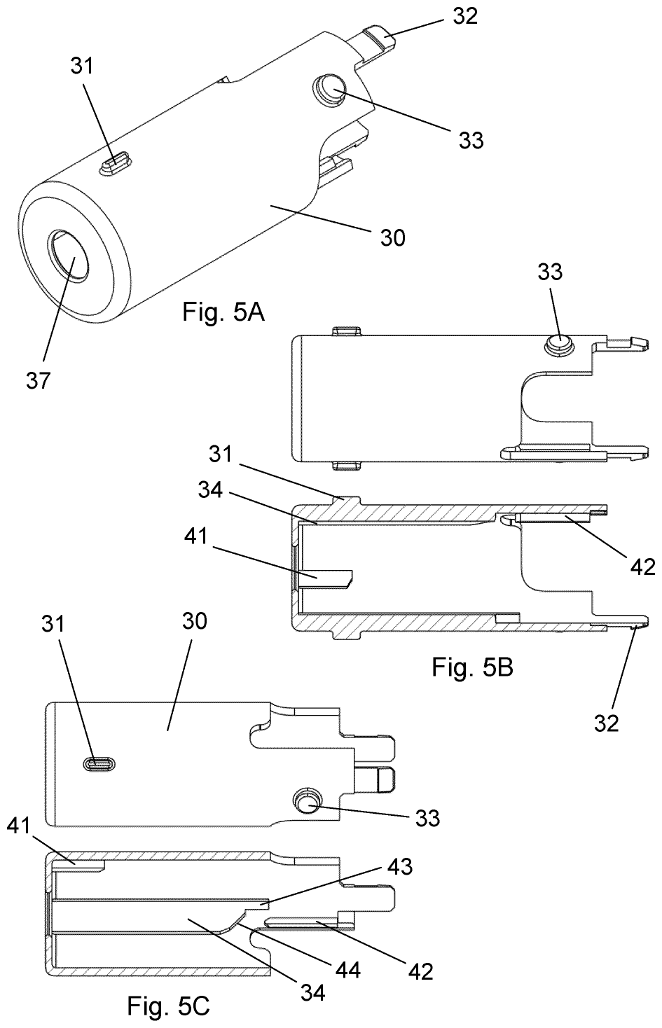

When the user rotates the protective cap 20 in order to remove it, these longitudinal tracks 22 engages similar outwardly pointing bars 31 provided on the outer surface of the first shield 30 as disclosed in FIG. 5A-C. Preferably, two such outwardly pointing bars 31 are provided.

The rotation of the protective cap 20 introduced by the peripheral track 12 is thus transferred to a similar rotation of the first shield 30.

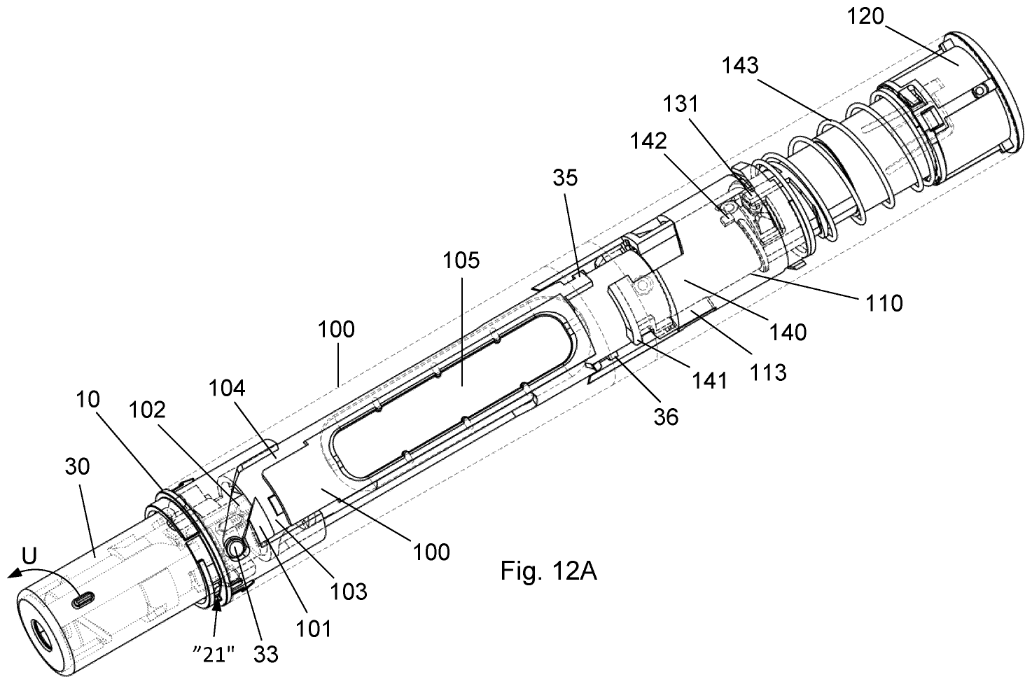

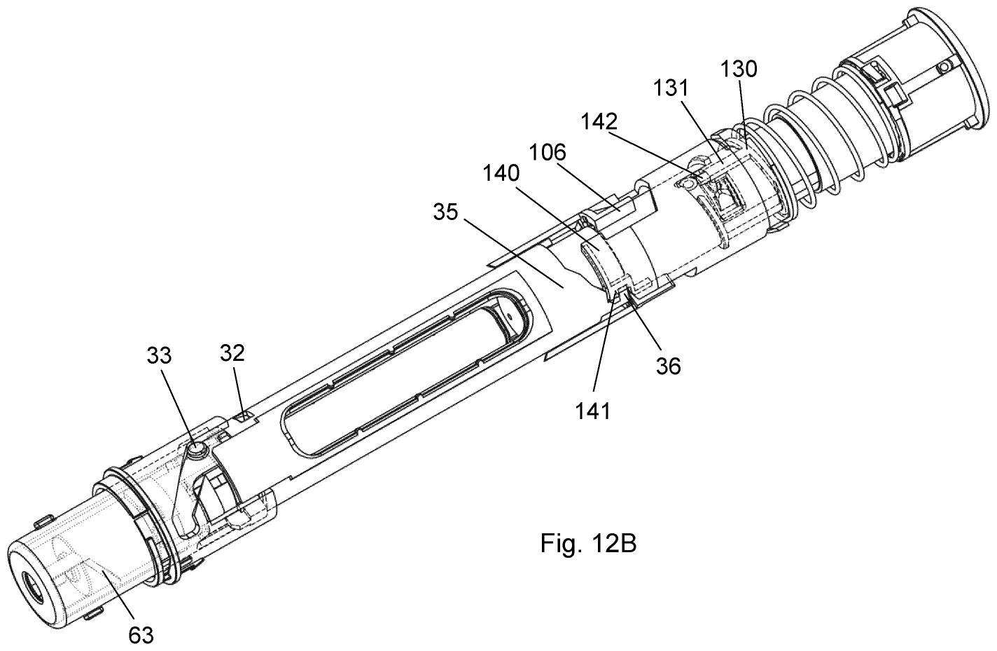

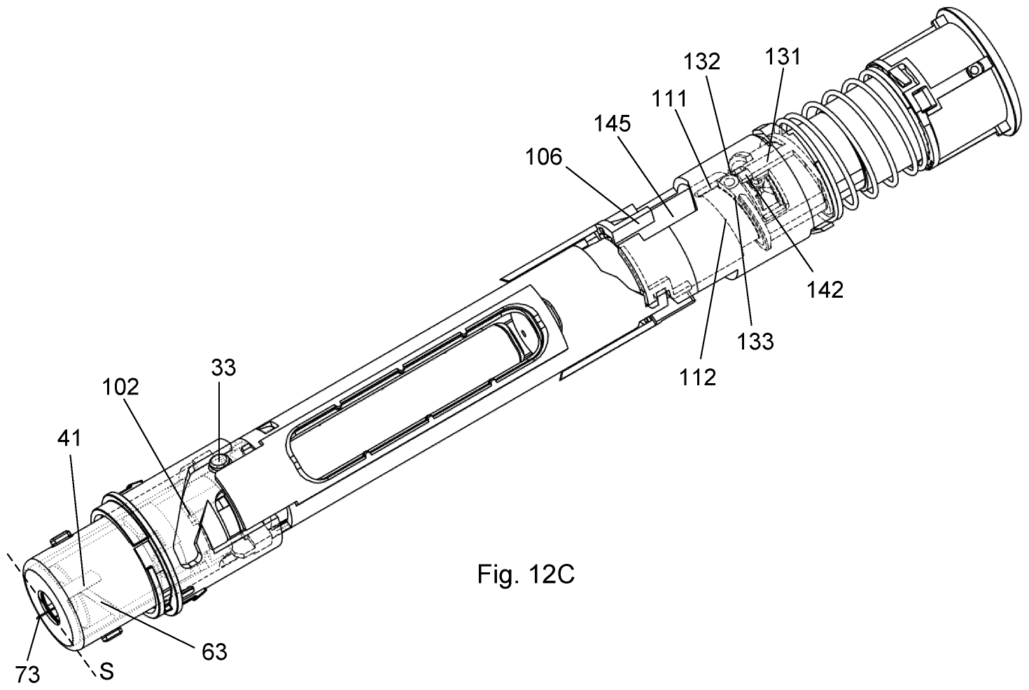

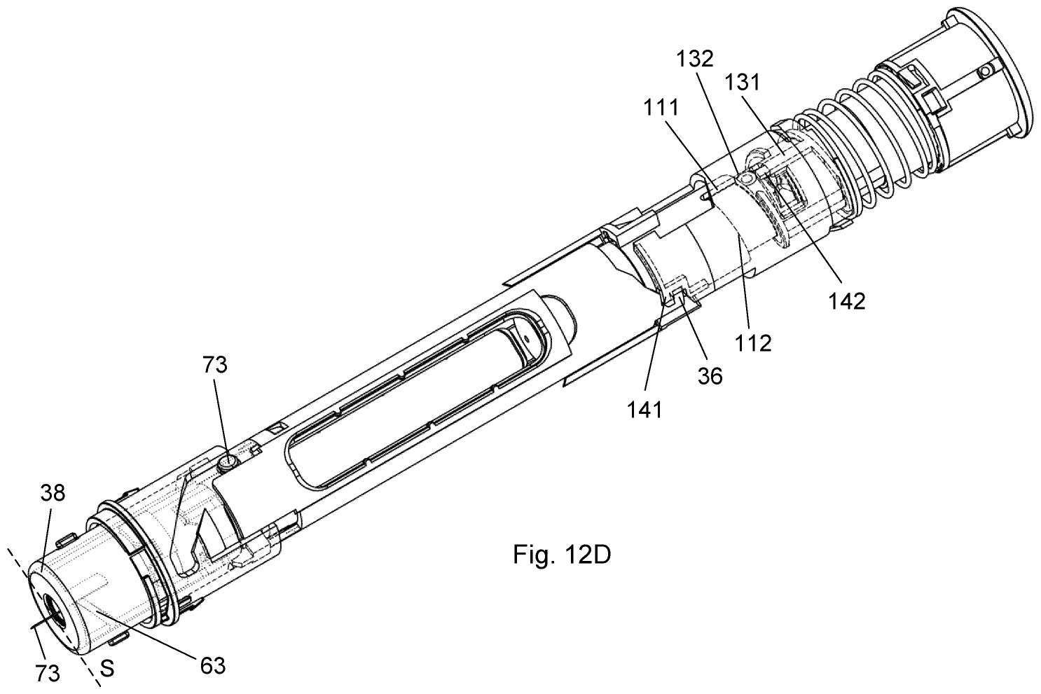

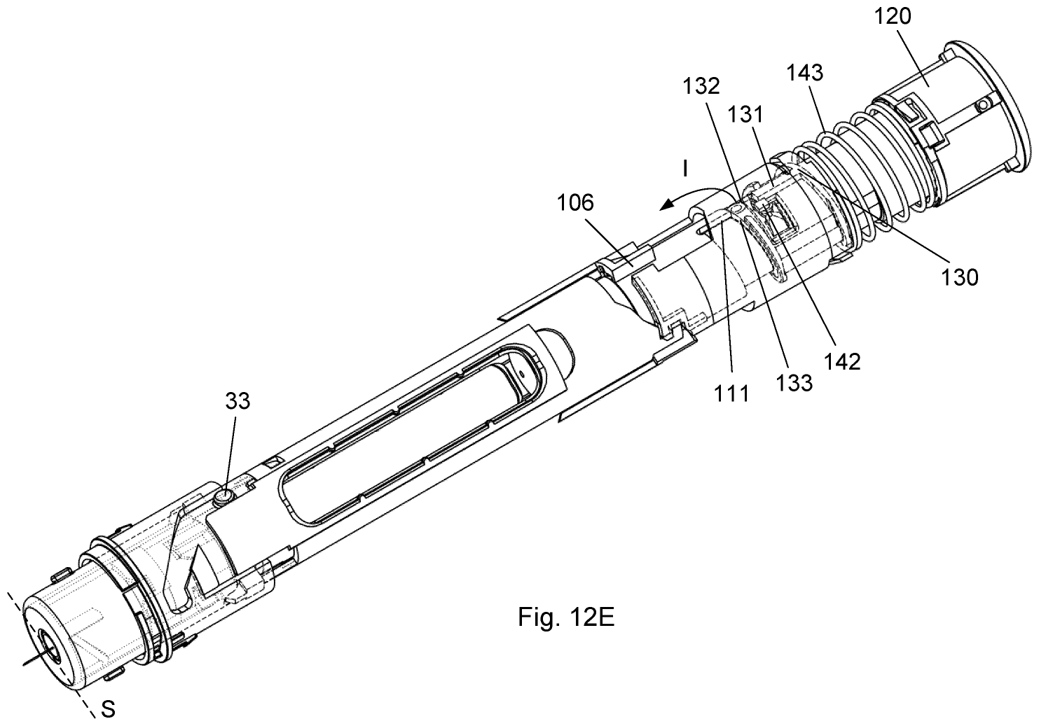

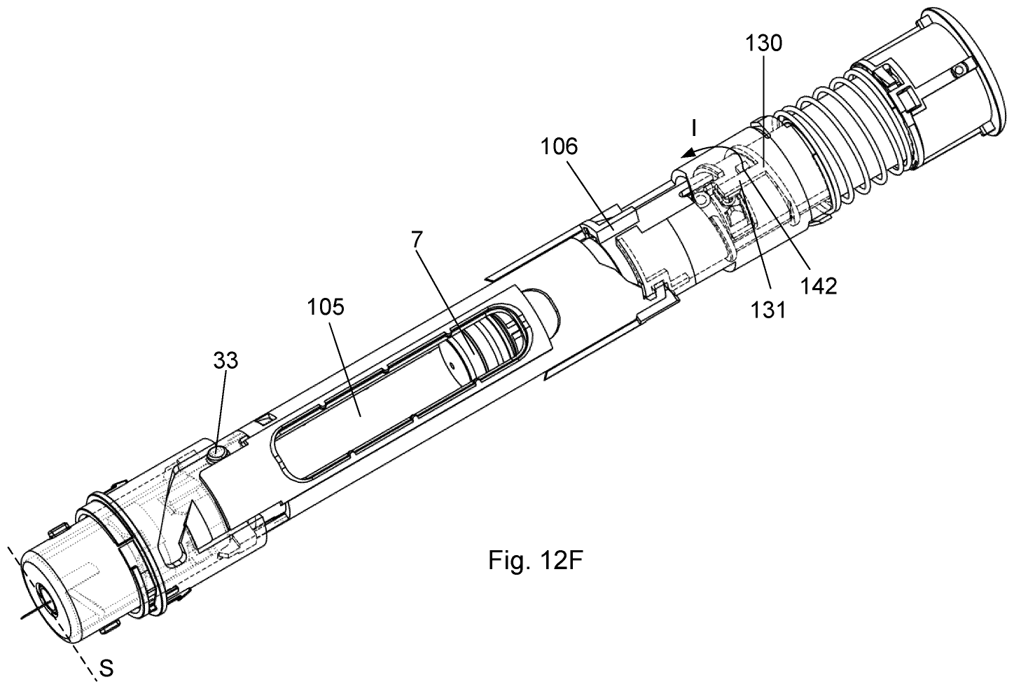

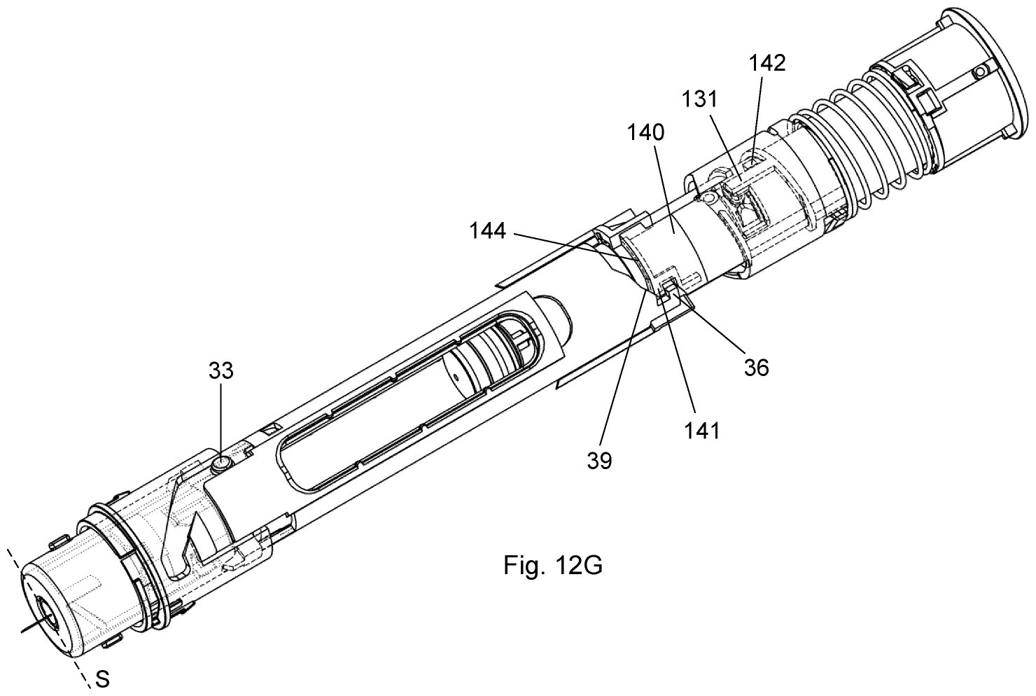

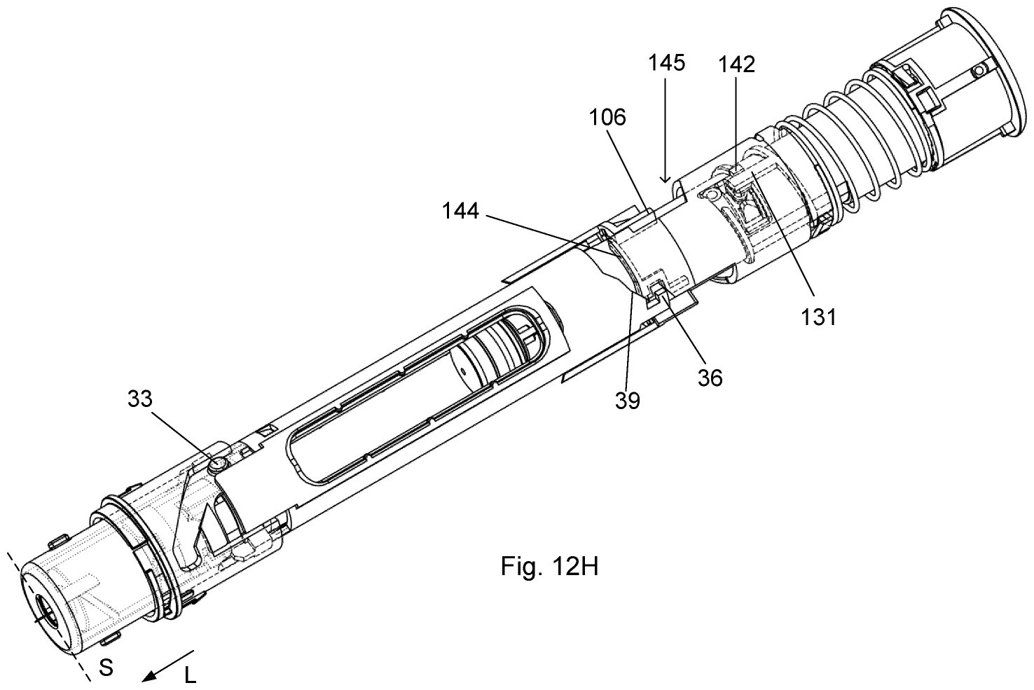

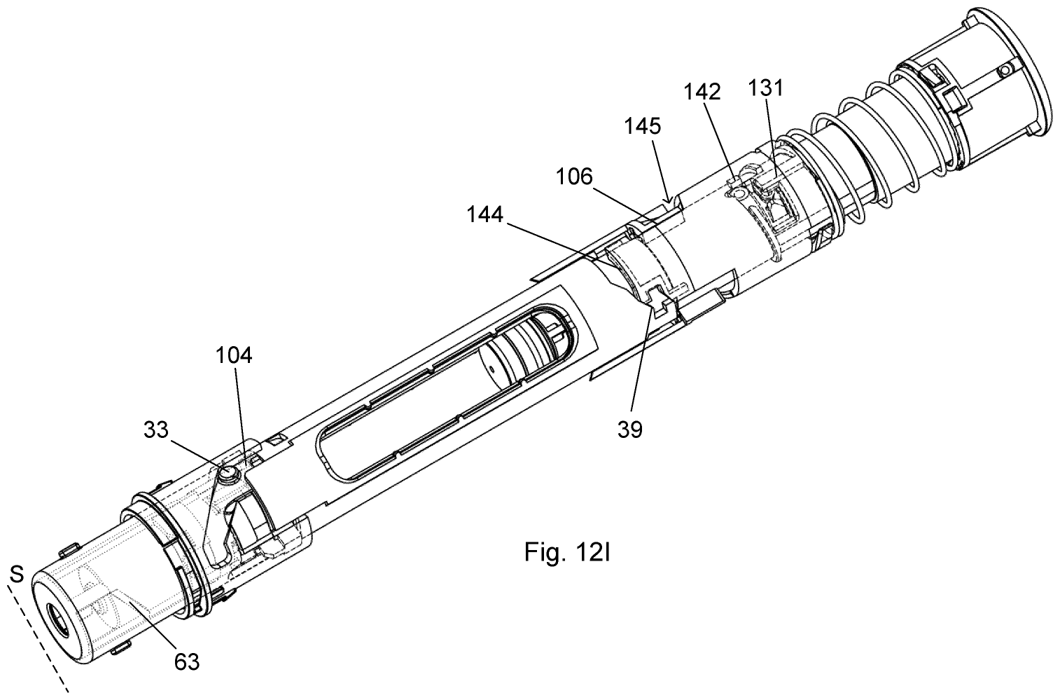

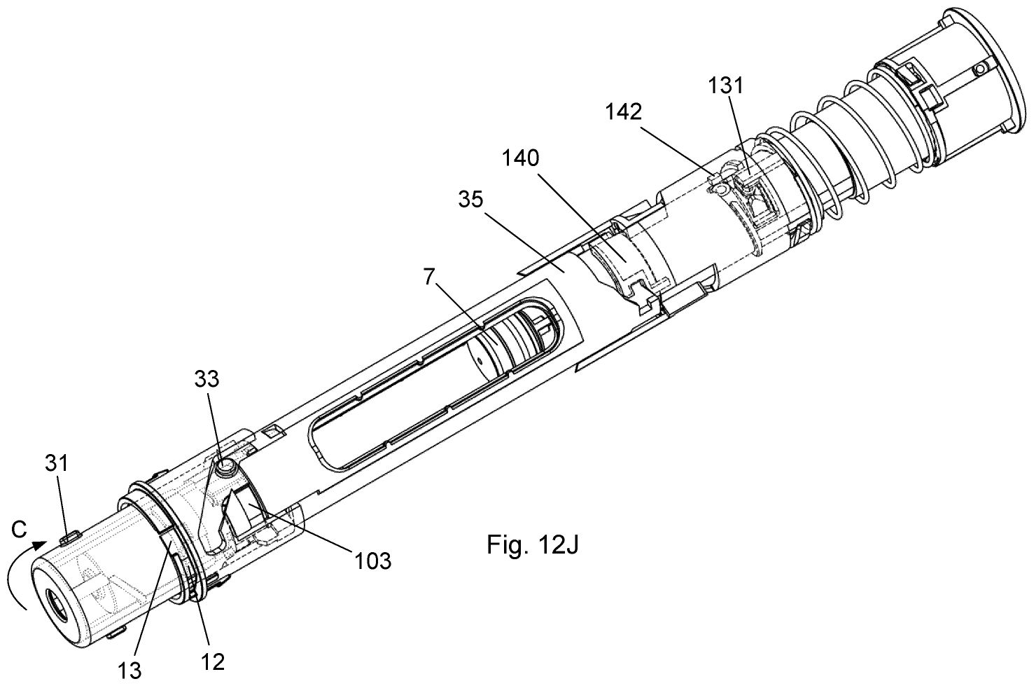

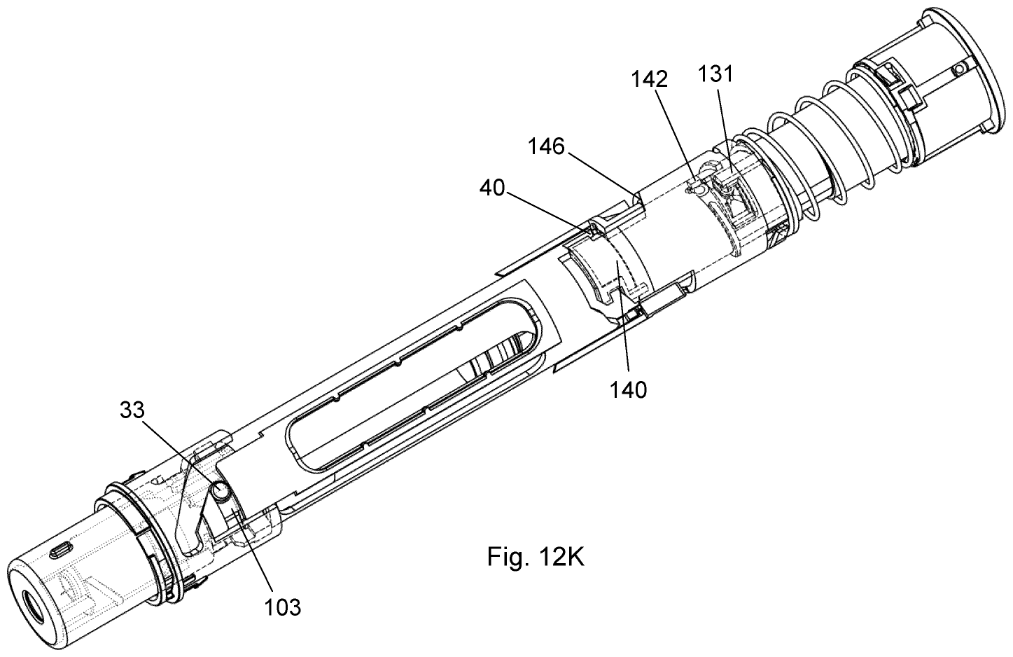

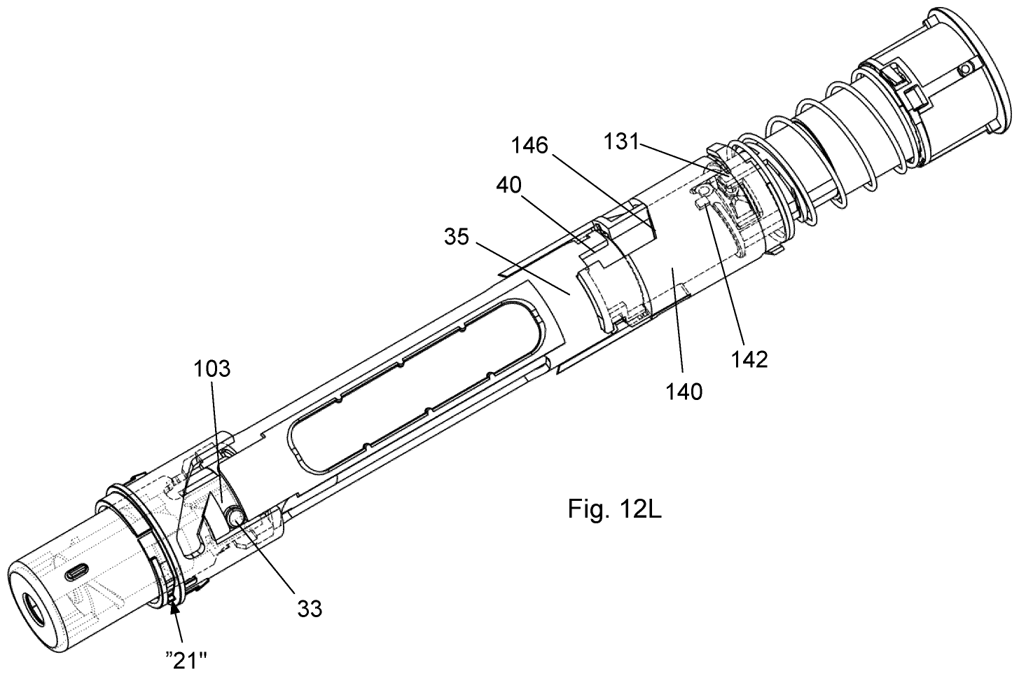

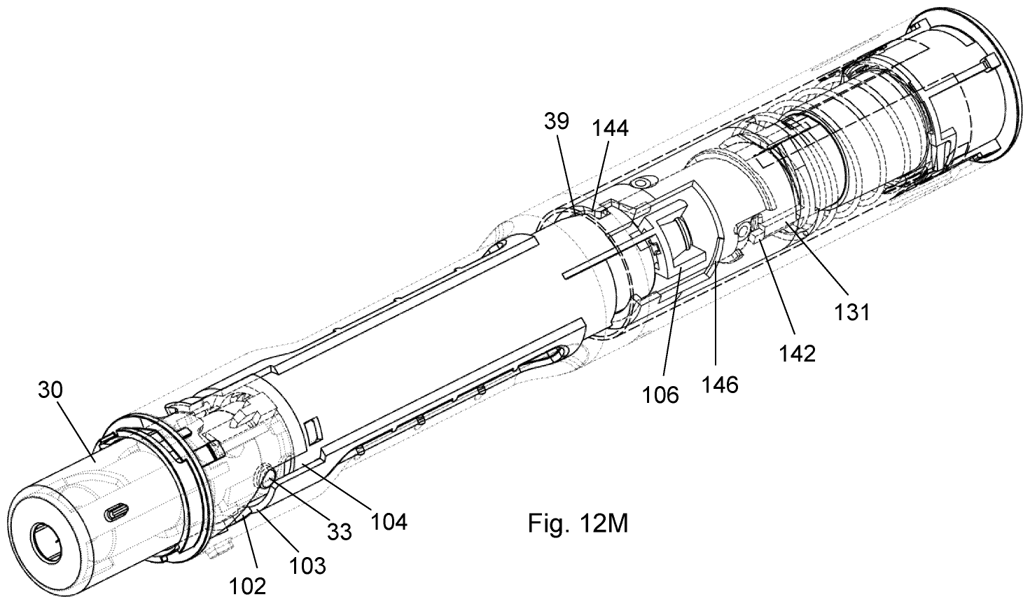

The first shield 30 disclosed in FIG. 5A-C is proximally provided with a pair of click arms 32 which are clicked onto a second shield 35 (see e.g. FIG. 12 B). This second shield 35 thus follows all movements of the first shield 30 both axially and rotationally. In an alternative example, the first shield 30 and the second shield 35 can be moulded as one unison element.

In the following the first shield 30 and the second shield 35 will together be named as the needle shield and referenced to as “30, 35”.

The first shield 30 is on the outer surface provided with a pair of protrusions 33 which together with guide surfaces 14 in the shield guide 10 makes the first shield 30 move helically when rotated as will be explained.

The guide surfaces 14 in the shield guide 10 connects to a helical guide part 101 provided in the housing part 100 such that a helical track 102 is formed between the shield guide 10 and the housing part 100 as e.g. seen in FIG. 12A-M. This helical track 102 is connected to a peripheral track 103 and to a longitudinal axial track 104 also provided on the inner surface of the housing structure. The use of these tracks will be explained later.

The injection device is disclosed in different states in the FIGS. 12A to 12L in which figures a part of the housing structure has been visually removed such that the second shield 35 can be at least partly seen.

The housing part 100 of the housing structure is further provided with the before mentioned window 105 through which the user can visually inspect the content of cartridge 6. The window 105 is preferably divided into sections indicating the number of predetermined dose volumes.

Distally, the needle cannula 72 is hidden by the first shield 30 which is connected to the second shield 35 as explained.

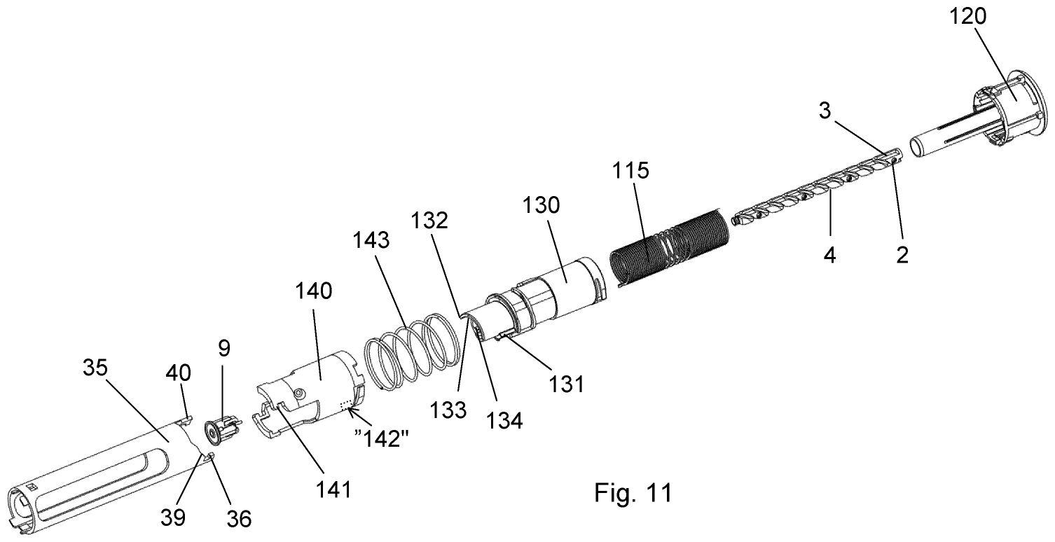

The housing part 100 which is thus only partially disclosed, is distally connected to the shield guide 10 and proximally connected to a spring base 120 as indicated with broken lines in FIG. 12A. Together the housing part 100, the shield guide 10, the cartridge holder part 5 and the spring base 120 makes up the housing structure. The spring base 120 further secures the proximal end of a torsion spring 115 which at its distal end is connected to a drive tube 130 as disclosed in FIG. 11.

The drive tube 130 engages and rotates a piston rod 2 during ejection as explained in International patent application No. PCT/EP2000/085271. In order to release one of the fixed doses, the user pushes the first shield 30 and consequently the second shield 35 in the proximal direction which moves the drive tube 130 proximally and releases the drive tube 130 to expel a fixed dose volume as explained in International patent application No. PCT/EP2000/085271.

When the drive tube 130 is released, the torsion spring 115 rotates the drive tube 130 and thus the piston rod 2 which is henceforth rotated helically in the distal direction due to its engagement with a nut element in the housing structure. In the disclosed example, the drive tube 130 is rotated approximately 360° every time it is proximally moved out of engagement with the housing part 100. The drive tube 130 engages a longitudinal track structure in the piston rod 2 such that the piston rod 2 rotate together with the drive tube 130. The piston rod 2 henceforth rotates simultaneously with, and the same angle as, the drive tube 30, and is moved helically forward a distance depending on the pitch of the thread on the piston rod 2 and the thread in the nut member in the housing part 100.

Preferably, a connector element 140 is provided for moving the drive tube 130 in the proximal direction during expelling of the fixed doses. The connector element 140 is provided between the second shield 35 and a connector spring 143 urging the connector element 140 in the distal direction. The second shield 35 and the connector element 140 are preferably hooked together in some situations as will be explained.

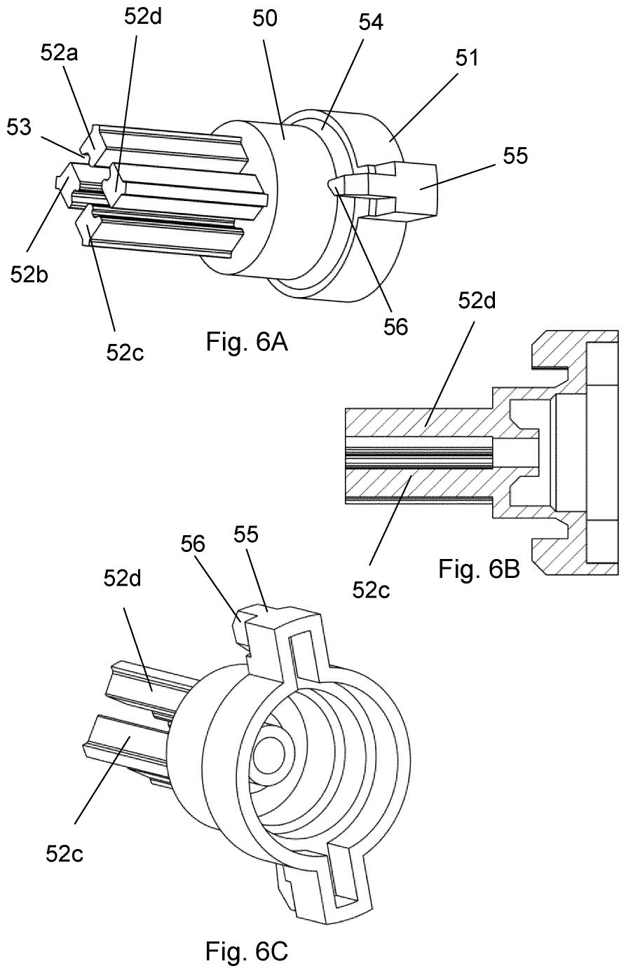

FIG. 6A-C discloses the tower 50 of the needle unit 45 and comprises a base part 51 and four uprights 52a,b,c,d. During initiation of the injection device, the base part 51 is connected to the housing structure such that the tower 50 can neither rotate nor move axially relatively to the housing structure during use of the injection device.

The uprights 52a,b,c,d are each provided with a longitudinal track structure 53 which engages the needle assemblies 70 as will be explained. The result is that the needle assemblies 70 can only move translationally in relation to the tower 50 and the housing structure. In the disclosed example the four uprights 52a,b,c,d are capable of guiding four needle assemblies 70 in the longitudinal direction. By using four needle assemblies it is assured that each of the four injections can be performed through a fresh and unused needle cannula 72.

However, the number of uprights and thus the number of longitudinal track structures can be changes to accommodate a different number of needle assemblies.

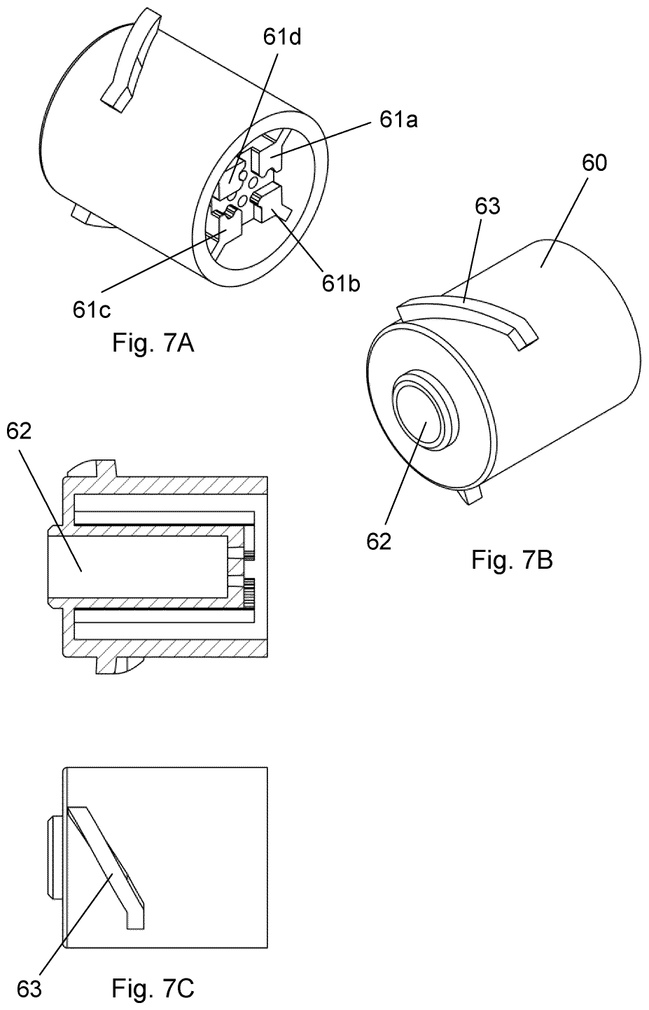

Distally the uprights 52a,b,c,d abuts four inserts 61a,b,c,d in the tip part 60 which are disclosed in details in FIG. 7A-C. These inserts 61a,b,c,d are moulded integrally with the tip part 60 and are connected to the distal end of the uprights 52a,b,c,d by gluing, welding or any alternative method , e.g. a click-fit. The tower 50 thus makes up the distal part, and the tip part 60 the proximal part of the needle unit 45 which is stationary maintained in the housing structure, both axially and rotational.

FIG. 7A-C further discloses the void 62 which secures the pierceable plug 65 and one or more helical ribs 63 provided on the outer surface of the tip part 60. The function of which will be explained.

A perspective view of the needle unit 45 disclosed in FIG. 2A is provided in FIG. 2B wherein the tower 50 is provided distally on the cartridge holder part 5 of the housing structure. Further, the uprights 52 on the tower 50 is permanently secured to the inserts 61 in the tip part 60 thus securing the tip part 60 to the tower 50. In FIG. 2B all the proximal ends 74 of the needle cannulae 72 has been penetrated into the cartridge 6 and is in contact with the liquid drug contained in the cartridge 6. A locking mechanism secures the tower 50 to the cartridge holder 5 after first activation as will be explained such that the needle unit 45 in the use position is physically secured to the housing structure.

In FIG. 2B is the outer contour of the needle shield 30 is shown in broken lines and the tip part 60 has been visually cut open to provide a better view of the needle assemblies 70

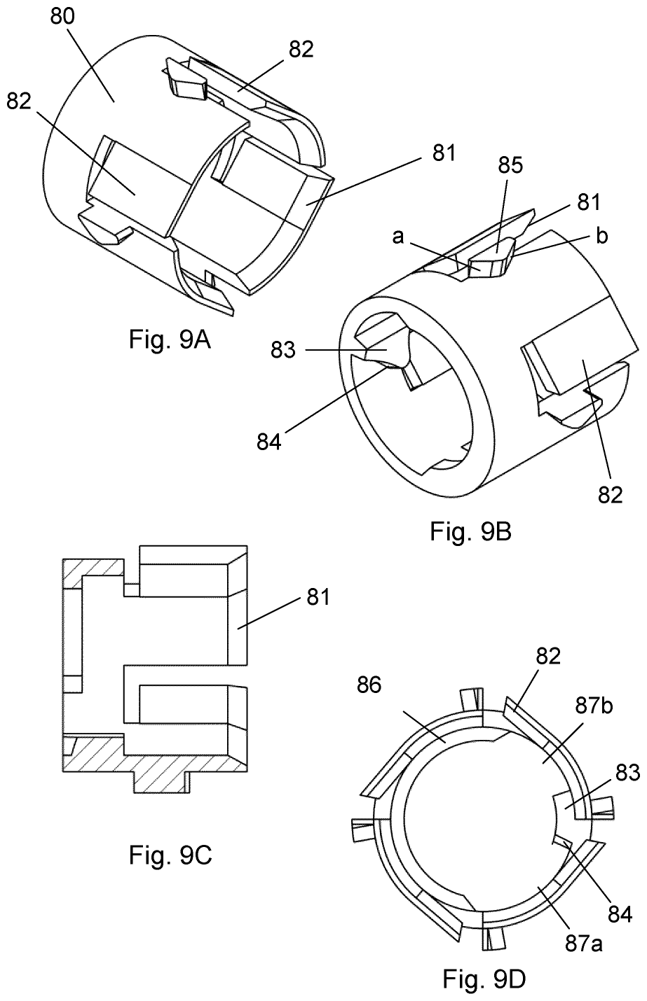

The key 80 which is disclosed in further details on FIG. 9A-D has a proximal end surface 81 rotatably provided on a sloped shoulder 54 on the tower 50 and are thus able to rotate relatively to the tower 50.

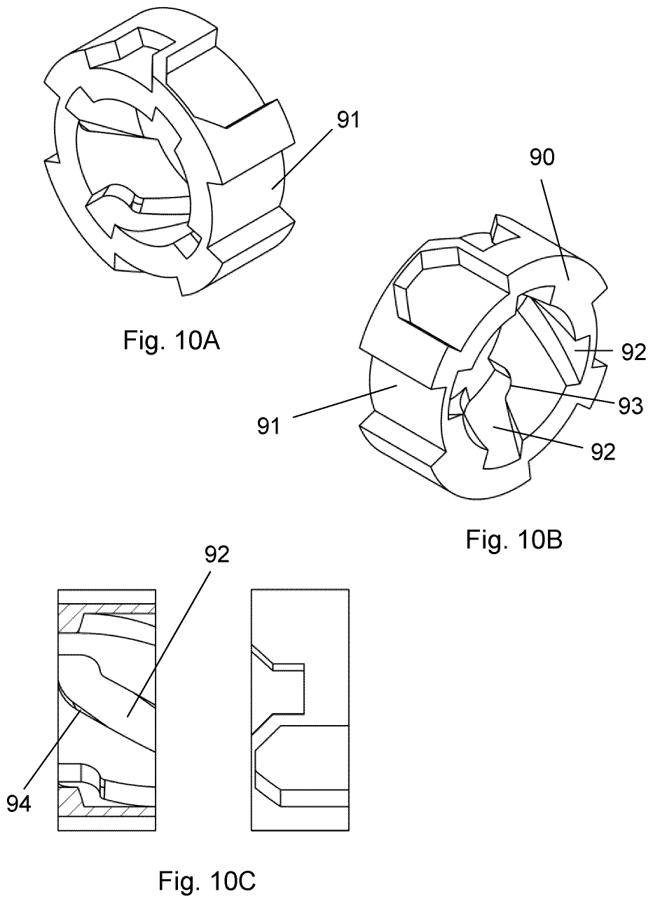

Between the key 80 and the tip part 60, a carousel 90 is provided. This carousel 90 is disclosed in further details in FIG. 10A-C. The carousel 90 is also rotatable in relation to both the tower 50 and the tip part 60. However, both the key 80 and the carousel 90 are prevented from axial movement in the longitudinal direction as the tower 50 and the tip part 60 are permanently secured to each other and sandwiched around the key 80 and the carousel 90.

The first shield 30 as disclosed in FIG. 5C, is on the inner surface provided with a number of longitudinal extending bars 34 which engage grooves 91 provided on the carousel 90 such that the carousel 90 is rotationally coupled to the first shield 30 and thus forced to rotate together with the first shield 30. As best seen in FIG. 2B these longitudinal bars 34 are able to slide axially in relation to the carousel 90.

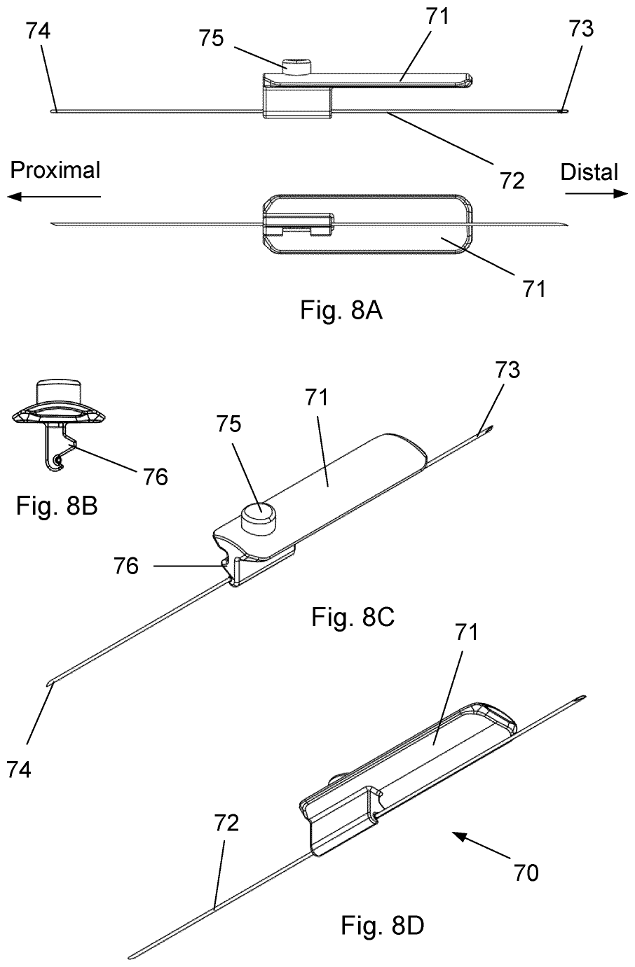

FIG. 8A-D discloses one example of the needle assemblies 70. Each needle assembly comprises a hub 71 which is moulded from a suitable polymer and holds a needle cannula 72.

Each needle cannula 72 has a distal end 73 and a proximal end 74 and a lumen there between. The needle cannula 72 is preferably glued and/or click fitted to the hub 71 and the hub 71 is further provided with a protrusion 75 on an outer surface.

The hub 71 is on the inwardly pointing surface provided with a rib configuration 76 which engage with the longitudinal track structure 53 in the uprights 52a,b,c,d on the tower 50 such that the needle assemblies 70 can only slide translational in relation to the needle unit 45 defined by the tower 50 and tip part 60. This also implies that the needle assemblies 70 can only slide translational in relation to the housing structure.

The working of the dose engine is described in the below example which is visualized on the FIGS. 12A to 12M wherein a total of four such needle assemblies 70 are provided. With four needle cannulae 72 being available also four fixed doses volumes should be available.

The dose engine itself is disclosed in an exploded view in FIG. 11 and comprises the spring base 120 which secures the proximal end of the torsion spring 115 to the remaining part of the housing structure. The opposite distal end of the torsion spring 115 is secured to the drive tube 130 such that the torque stored in the torsion spring 115 is applied to a rotation of the drive tube 130 as will be explained.

The drive tube 130 is proximally provided with a helical end surface 133 which terminates into an axial drive flange 132 extending in parallel with the centre axis “X” of the injection device. Further, an axial rib 131 is provided on an outer surface of the drive tube 130. The drive tube 130 is also described in further details in International patent application No. PCT/EP2000/085271. The centre of the drive tube 130 comprises a through-going opening which surrounds the piston rod 2.

The piston rod 2 has a longitudinal track structure 3 and an outer thread 4. The longitudinal track structure 2 is engaged by one or more radial teeth 134 provided in the through-going opening the drive tube 130, and the outer thread 4 on the piston rod 2 is threaded to a similar thread in the housing structure. The result being that the piston rod 2 is moved helically in relation to the housing structure whenever the drive tube 130 is rotated.

In the example disclosed on FIG. 11, a piston rod foot 9 can be provided to better distribute the force onto the plunger 7 inside the cartridge 6 during dose expelling.

To move the drive tube 130 in the proximal direction during dose activation a connector element 140 is provided which lies between the drive tube 130 and the needle shield 30, 35.

The connector element 140 is urged in the distal direction by a connector spring 143 which is provided between the spring base 120 and the connector element 140 and applies an axial force onto the connector element. Distally the connector element 140 is provided with a number of hooks 141 which are able to engage with similar hooks 36 provided proximally on the second shield 35 as will be explained.

### Operation of the Injection Device in Regards of Expelling the Doses

As seen in the FIGS. 12A-M, the first shield 30 is clicked to the second shield 35 by the click arm 32 engaging the second shield 35 such that these two elements operate as one needle shield 30, 35. This is especially clear from FIG. 12B. The second shield 35 is further proximally provided with a hook 36 which is able to engage with a similar hook 141 provided distally on the connector 140.

In FIG. 12A-M, the housing part 100 is provided with an internal bridge structure 110 which is disclosed with broken lines. This bridge structure 110 is preferably moulded integrally with the housing part 100. The bridge structure 110 is separated from the housing part 100 by a number of carriers 113 which allows the connector element 140 to move axially. The bridge structure 110 is further, at a proximal end, provided with an axial housing flange 111 as also disclosed in International patent application No. PCT/EP2000/085271.

On the inner surface, the connector element 140 is provided with an inwardly pointing knob 142 which thus follows all movements of the connector 140. In the FIGS. 12A-M, this inwardly pointing protrusion 142 is disclosed with broken lines, such that its movement in the different states is visualized. The inwardly pointing protrusion 142 is also indicated with broken lines in FIG. 11. Preferably, two inwardly pointing protrusions 142 are provided as disclosed in International patent application No. PCT/EP2000/085271, however, in the example disclosed herein only one is shown.

The inwardly pointing knob 142 operates a longitudinal rib 131 provided on the drive tube 130 during dose expelling as will be explained.

The drive tube 130 further has an axial drive flange 132 which abut the axial housing flange 111 inside the housing part 100 as disclosed in FIG. 12A-D. When the axial drive flange 132 is moved axially out of engagement with the axial housing flange 111 as seen in FIG. 12E, the torsion spring 115 will rotate the driver tube 130 one full rotation i.e. 360° back to its initial position (FIG. 12G). During this rotation, the drive tube 130 will rotate the piston rod 2 which is thus moved helically forward in the distal direction.

In the FIGS. 12A-M only one axial housing flange 111 and one axial drive flange are disclosed. However, two of each of the flanges as disclosed in International patent application No. PCT/EP2000/085271 are preferred.

The pair of protrusions 33 provided on the first shield 30 is movable in the helical track 102, in the axial track 104 and in the peripheral track 103. The pattern of movement of the first shield 30 are the same as the pair of protrusions 33 as they are provided on the first shield 30. The position and the movement of the pair protrusions 33 in these tracks 102, 103, 104 defines the different states of the injection device as will be explained further.

The doses are released by moving the first shield 30 and thus also the second shield 35 in the proximal direction. This is preferably done by pressing the distal end of the first shield 30 against the skin of the subject to be injected. The proximal movement of the first shield 30 and the second shield 35 is via the connector element 140 transferred to a proximal movement of the drive tube 130. The axial length of the housing flange 111 and the axial drive flange 132 is henceforth decisive for how long the drive tube 130 most be moved in the proximal direction before a dose is released, and the pitch between the piston rod 2 and the nut member in the housing structure decides how far the piston rod 2 is rotated helically in the distal direction during release of the dose. Since these factors are the same for all the doses provided for, the piston rod 2 moves the same axial distance in the distal direction for each dose release why all the doses are identical in volume. However, minor variations can occur due to tolerances.

It is further to be understood that once the drive tube 130 is moved proximally and the axial drive flange 132 is moved out of engagement with axial housing flange 111, the helical end surface 133 of the drive tube 130 will rotate on the helical structure 112 of the bridge structure 110 inside the housing structure. The force providing this rotation is delivered by the torsion spring 115 which operates between the housing structure and the drive tube 130. The rotation of the drive tube 130 stops once the axial drive flange 132 abuts against the housing flange 111 which in the disclosed example means that the drive tube 130 rotates one full rotation i.e. 360° each time the drive tube 130 is activated to deliver one dose.

The torsion spring 115 is preferably loaded with a torque sufficient to rotate the drive tube 130 a predefined number of times to expel the same predefine number of dose volumes. In the example herein provided, the multi-use fixed dose device is designed to expel four equally sized dose volumes and the torsion spring 115 is thus pre-strained to deliver a torque sufficient to rotate the drive tube 130 four times. However, any other random number of fixed doses can be envisaged, e.g. 2, 3, 5, 6 or more fixed dose volumes and the torque stored in the torsion spring is preferably set to the chosen number of dose volumes. Do to this pre-straining of the torsion spring 115 it is not necessary for the user to manually strain the torsion spring 115 every time a new dose is to be expelled.

The fixed doses to be expelled are thus prepared by moving the axial drive flange 132 on the drive tube 130 axially out of engagement with the axial housing flange 111 and the fixed dose is expelled when the drive tube 130 rotates one full rotation down the helical structure 112 provided on the bridge structure 110 inside the housing structure. For each dose release, the piston rod 2 moves a certain, and pre-determined, distance in the distal direction and as explained in International patent application No. PCT/EP2000/085271 and a stop mechanism incorporated between the drive tube 130 and the piston rod 2 prevents the drive tube 130 from being moved further in the proximal direction when the last of the predetermined number of fixed doses has been prepared.

When the pair of protrusions 33 is provided in the start of helical track 102 (FIG. 12A) it is not possible to translate the first shield 30 in the proximal direction and since the first shield 30 cannot be translated proximally it is not possible to release any of the fixed doses while the first shield 30 is in a rotational position wherein the pair of protrusions 33 are positioned in the start of the helical track 102. The first shield 30 is henceforth locked against translational movement in the position disclosed in FIG. 12A.

Further, in the state disclosed in FIG. 12A, the protective cap 20 would cover the first shield 30 but in order to visualize the way of operation, the protective cap 20 is not shown in FIG. 12A.

The different states correlated with the different positions of the pair of protrusions 33 are described in further details below. In the FIGS. 12A-M underlying structures are shown with broken lines. The sequences or states to be explained are:

1. Unlocking the injection device for First Dose.

2. Preparing the First Dose.

3. Injecting the First Dose.

4. Sliding back the Needle Shield After First Injection.

5. Attachment of the Protective Cap Following First Injection and locking of the injection device.

6. Unlocking the Injection Device for Next Dose.

7. Injection of the Next Doses.

### Unlocking the Injection Device for First Dose

### FIG. 12A-B

FIG. 12A discloses the injection device as delivered to the user. In order to better visualize the injection device, the injection device disclosed in FIG. 12A is shown without the protective cap 20, however when delivered to the user, the inner protrusion 21 inside the protective cap 20 would be located in its locked position in the peripheral track 12 on the shield guide 10, this position is indicated with the arrow “21” in FIG. 12A.

When the user rotates the protective cap 20 counter clock-wise to remove it, this rotation will be transformed to a similar rotation of the first shield 30 indicated by the arrow “U” in FIG. 12A. This rotation will move the pair of protrusions 33 on the first shield 30 from the locked position disclosed on FIG. 12A to the open position disclosed in FIG. 12B.

In the locked position disclosed in FIG. 12A, the first shield 30 cannot be translated in the proximal direction as the pair of protrusions 33 is hindered in proximal movement by the proximal side of the helical track 102. The helical track 102 is most distally provided with a more peripheral shape which enhances the resistance for the pair or protrusions 33 to move in the proximal direction. However, once the first shield 30 has been rotated to the unlocked position shown in FIG. 12B, the pair of protrusions 33 can slide freely in the axial track 104 and the first shield 30 can be translated in the proximal direction.

During rotation of the first shield 30 from the position in FIG. 12A to the position disclosed in FIG. 12B, the first shield 30 together with the second shield 35 first rotates and moves helically in the proximal direction due to the helical guidance of the pair of protrusions 33 in the helical track 102. The first part of the movement brings the hook 36 provided proximally on the second shield 35 into engagement with the hook 141 on the connector element 140 and on the last part of the rotation of the needle shield 30, 35, the connector 140 is also rotated helically into the position shown in FIG. 12B.

This further helical rotation of the needle shield 30 and the second shield part 35 is thus transferred to a similar helical rotation of the connector element 140 which moves the inwardly pointing knob 142 inside the connector element 140 into alignment with the longitudinal rib 131 provided on the outer surface of the drive tube 130 as seen in FIG. 12B. The first shield 30 is thus unlocked and an injection can hereafter be performed by pressing the distal end of the first shield 30 against the skin of the subject to be injected.

In the unlocked position of the needle shield 30, 35 disclosed in FIG. 12B, the connector 140 is slidable along a raised bar 106 provided inside the housing structure.

Before an injection can be performed the user must remove the protective cap 20. In the position disclosed in FIG. 12B, the inner protrusion 21 is aligned with the track opening 13 and the protective cap 20 can be axially pulled off the housing structure.

### Preparing the First Dose

### FIG. 12B-C-D

In FIG. 12B, the protective cap 20 has been removed and the rotation of the protective cap 20 and hence the first shield 30 has moved the pair of protrusions 33 to a position in the start of the axial track 104. The first shield 30 has thus been rotated from the locked position to the unlocked position and an injection can be performed by pressing the distal end of the first shield 30 against the skin (indicated by the broken line “S”) of the subject to receive the injection.

The inwardly pointing knob 142 on the connector element 140 is in this state positioned adjacent to and in abutment with the longitudinal rib 131 on the drive tube 130, and the second shield 35 is hooked 36, 141 to the connector element 140.

As the first shield 30 are pressed further against the skin “S” as disclosed in FIG. 12C, the first shield 30 moves translational in the proximal direction (guided in the axial track 104) and the distal end 73 of the needle cannula 72 starts appearing through the distal opening 37 in the front end 38 of first shield 30 and penetrates into the skin “S” of the subject being injected.

At the same time, the connector element 140 moves translational in the proximal direction guided by the raised bar 106 and due to the abutment between the inwardly pointing knob 142 and the distal end of the longitudinal rib 131, the drive tube 130 is also moved proximally.

During this proximal movement of the drive tube 130, the axial drive flange 132 on the drive tube 130 slides proximally on the axial housing flange 111 provided on the bridge structure 110. However, as long as the axial drive flange 132 abuts the axial housing flange 110, the drive tube 70 is kept inrotatable.

The axial movement of the drive tube 130 compresses the compression zone provided on the torsion spring 115. This compression zone is best seen on FIG. 11.

In FIG. 12D, the first shield 30 has almost reached its most proximal position. This is reflected in the close proximity between the inner part of the front end 38 on the first shield 30 and the helical rib 63 provided on the outside of the tip part 60 meaning that the pre-determined dose volume is about to be released. In this position of the needle shield 70, the distal end 73 of the needle cannula 72 is usually inserted around 2 to 8 mm into the skin of the subject to be injected and the injection i.e. the transport of liquid drug through the lumen of the needle cannula 72, can start. Further, in the position shown on FIG. 12D is the drive flange 132 almost moved past and out of engagement with the housing flange 111 which thus is about to set the torsion spring 115 free to start to rotate the drive tube 130.

### Injection of First Dose

### FIG. 12E-F-G

As seen in FIG. 12E, the drive tube 70 has been moved axially to a position wherein the axial drive flange 132 is out of engagement with the axial housing flange 111 such that the torsion spring 115 will start to rotate the drive tube 70 which is indicated by the arrow “I” in FIG. 12E and FIG. 12F. The drive tube 130 is guided to move helically (by the engagement of the helical end surface 133 with the helical structure 112) in the distal direction until the axial drive flange 132 again encounters the axial housing flange 111 as explained in further details in International patent application No. PCT/EP2000/085271. The rotation of the drive tube 70 is transferred to a similar rotation of the piston rod 2 which moves helically in the distal direction and pushes the plunger 7 forward the predetermined distance inside the cartridge 6 such that the fixed dose volume is expelled.

The drive tube 130 (see e.g. FIG. 11) has an inner opening which surrounds the piston rod 2 and this inner opening is provided with one or more radial teeth 134 engaging a longitudinal track structure 3 in the piston rod 2 such that the piston rod 2 is forced to rotate together with the drive tube 130. The nature of the engagement between the drive tube 130 and the piston rod 2 is such that the piston rod 2 is able to move axially relatively to the drive tube 130. During its rotation, the piston rod 2 is screwed helically forward in the distal direction due to its threaded engagement with the nut member associated with the housing structure. Such drive mechanism is well-known from many different injection devices. In the embodiment described herein, the drive tube 130 and the piston rod 2 is rotated one full rotation i.e. 360° for each dose release. The axial distance the piston rod 2 is moved in the distal direction during such full rotation depend on the pitch on the thread connection between the piston rod 2 and the nut member in the housing structure.

The nut member facilitating the rotation of the piston rod 2 is preferably carried by the housing structure. The nut member is either permanently attached e.g. by moulding it to the bridge structure 110 or the nut member can be attached thereto using alternatives means.

During the rotation of the drive tube 130 from the position in FIG. 12E to the position in FIG. 12F, the drive tube 130 moves helically during dose expelling whereas the connector element 140 carrying the inwardly pointing knob 142 remains axially stationary during dose expelling since the first shield 30 is pressed against the skin of the subject to be injected, the first shield 30 and the second shield 35 are thus kept stationary during injection. The connector element 140 is henceforth axially locked between the connector spring 143 and the second shield 35 and thus also kept stationary.

FIG. 12F discloses the situation in which the first fixed dose volume has almost been fully delivered and as indicated by the arrow “I”, the drive tube 130 is still rotated by the torsion spring 115. The distal end 73 of the needle cannula 72 remains inserted into the skin of the subject being injected and the plunger 7 has now moved to the first indication in the window 105 on the housing structure which in this example indicates that one fixed dose has been expelled and three (3) doses remains. The drive tube 130 has been rotated approximately one full rotation of 360° by the torsion spring 115 and the drive tube 130 has been moved helically in the distal direction. In this position, the axial drive flange 132 approaches and almost abuts the axial housing flange 111, and the side surface of the longitudinal rib 131 which has also been rotated one full rotation abuts the inwardly pointing knob 142, however on the opposite side as disclosed in FIG. 12E since the drive tube 130 has rotated in the counter clock-wise direction from the position in FIG. 12E to the position in FIG. 12F.

In FIG. 12G, the axial drive flange 132 engages the axial housing flange 111 and the first fixed dose has been fully delivered. In this position, the longitudinal rib 131 on the drive tube 130 has pushed and rotated the inwardly pointing protrusion 142 and henceforth the connector element 140 in the counter clock-wise direction and the hook 141 on the connector element 140 has started to uncouple from the hook 36 on the second shield 35.

The rotation of the connector element 140 also moves the end flange 144 of the connector element into contact with a sloped flange 39 proximally on the second shield 35. However, as the first shield 30 and the second shield 35 is in their most proximal position and the skin of the subject being injected hinders the first shield 30 from moving in the distal direction this has no consequence for the connector element 140 which thus remains in the same axial position.

Sliding back the Needle Shield after First Injection

### FIG. 12H-I

In FIG. 12H, the user starts to remove the first shield 30 from the skin of the subject being injected such that the needle shield 30,35 slides in the distal direction as indicated by the arrow “L” in FIG. 12H. In this situation both the second shield 35 and the connector element 140 will start to move in the distal direction due to the connector spring 143 applying a force onto the connector element 140. The inwardly pointing knob 142 on the connector element 140 will thus slide along on the longitudinal rib 131 on the drive tube 130 in the proximal direction as the drive tube 130 will remain in its position abutting the bridge structure 110 inside the housing structure. This abutting position was reached on FIG. 12G.

Since the second shield 35 is prevented from rotation due to the engagement of the pair of protrusions 33 on the first shield 30 with the axial track 104, the abutment of the end flange 144 on the connector element 140 with the sloped flange 39 on the second shield 35 will force the connector element 140 to rotate further in the counter clock-wise direction as it moves distally and releases from the raised bar 106 (FIG. 12I). The hook 141 on the connector element 140 will thus start to disengage the hook 36 on the second shield 35.

In FIG. 12I, the distal part 73 of the needle cannula 72 and the first shield 30 has been fully removed from the skin of the subject receiving the injection. In this position, the pair of protrusions 33 on the first shield 30 is positioned at the distal end of the axial track 104 and the first shield 30 fully covers the distal end 73 of the needle cannula 72.

At the same time; the engagement between the end flange 144 on the connector element 140 and the sloped flange 39 on the second shield 35 has rotated the connector element 140 further to a position wherein a carved-out portion 145 on the connector element 140 engages a raised bar 106 in the housing structure such that the connector element 140 is now hooked and secured to the housing structure.

In this position, the connector element 140 is prevented from being moved translational in the proximal direction due to the engagement between the carved-out portion 145 and the raised bar 106. It is henceforth not possible to move the needle shield 30, 35 in the proximal direction. Should the user try to activate a new dose when in this state it will not be possible to move the needle shield 30, 35 proximally as the connector element 140 is locked in the proximal direction. The first shield 30 is consequently locked from any movement in the proximal direction.

The inwardly pointing knob 142 is in this position located counter clockwise to the rib 131 such that should the connector 140 be moved proximally in this state, it will not release a further dose.

### Attachment of the Protective Cap Following First Injection

### FIG. 12J-K-L

When the injection is finished, the user attaches the protective cap 20 by inserting the inner protrusion 21 on the protective cap 20 into the peripheral track 12 on the shield guide 10 through the axial opening 13 in the peripheral track 12 and starts to rotate the protective cap 20 in the clock-wise direction. All though the protective cap 20 is not shown in FIG. 12J, the rotational movement delivered by the protective cap 20 is illustrated by the arrow “C”.

This rotation is transferred to a similar rotation of the first shield 30 due to the engagement between the longitudinal ribs 22 inside the protective cap 20 and the outwardly pointing bars 31 on the first shield 30.

In FIG. 12K, the continued rotation of the protective cap 20 and henceforth the first shield 30 has moved the pair of protrusions 33 further into the peripheral track 103. Also, in this position an axial flange 40 on the second shield 35 abuts the connector element 140 and starts to rotate the connector element 140 in the clock-wise direction.

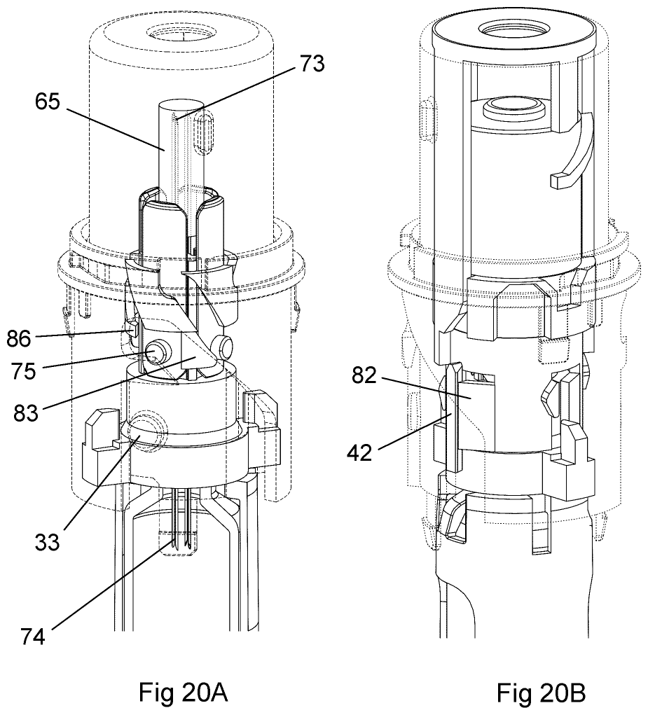

The carved-out portion 145 in the connector element 140 is proximally provided with a sloped flange 146. Consequently, when the connector element 140 is rotated in the clock-wise direction as disclosed in FIG. 20, this sloped surface 146 in combination with the spring 143 urges the connector element 140 in the distal direction ensuring that the connector element 140 moves helically in the distal direction.

Once the protective cap 20 has been rotated to its locked position in the peripheral track 12, the pair of protrusions 33 are located in the clock-wise end of the peripheral track 103 as shown in FIG. 12L and the injection device is fully locked. In this position, the inner protrusion 21 inside the protective cap 20 is again positioned in the bottom of the peripheral track 12 which position is indicated by the arrow marked “21” in FIG. 12L.

As also seen in FIG. 12L, the connector 140 has entered into engagement with the second shield 35 and the inwardly pointing protrusion 142 inside the connector 140 is positioned a few degrees clock-wise to the rib 131 on the drive tube 130.

### Unlocking the Injection Device for Next Dose

### FIG. 12M

Whenever the user wants to perform a new injection, the user must remove the protective cap 20 by rotating the inner protrusion 21 to a position wherein it is aligned with the radial opening 13 in the peripheral track 12 and remove the protective cap 20 as seen in FIG. 12M. Such rotation also rotates the pair of protrusions 33 through the peripheral track 103 to the position in the axial track 104 disclosed in FIG. 12M.

In FIG. 12M, the injection device is shown as rotated approximately 90 when compared to the views in FIG. 12A-L. A part of the housing structure has been visually cut away to enhance the view and some underlying structures are shown in broken lines.

When rotating the second shield 35 from the position disclosed in FIG. 12L to the position disclosed in FIG. 12M, the engagement between the sloped flange 146 on the connector 140 and the raised bar 106 in the housing structure forces the connector 140 to move helically in the proximal direction and when the sloped flange 39 on the second shield 35 abuts the end flange 144 on the connector 140, the connector 140 is moved into the position disclosed in FIG. 12M. This movement can also be facilitated by an additional guiding track provided in the housing structure.

When unlocking the injection device by rotating the needle shield 30 (using the protective cap 20) from the position disclosed in FIG. 12L to the position disclosed in FIG. 12M the following happens:
The inner protrusion 21 on the protective cap 20 is moved to the radial opening 13 in the peripheral track 12 and the protective cap 20 is removed.
The pair of protrusions 33 on the first shield 30 are moved from the bottom of the peripheral track 103 and into the axial track 104. This means that the injection device is once again unlocked and ready to perform an injection.
The connector 140 is moved helically by the engagement between the sloped flange 146 on the connector 140 and the raised bar 106 in the housing structure.
The connector 140 is moved further in the proximal direction by the engagement of the end flange 144 on the connector 14 and the sloped flange 39 on the second shield 35 which is rotated.

The result being that the inwardly pointing protrusion 142 provided on the inner surface of the connector 140 is moved into alignment and abutment with the rib 131 provided on the drive tube 130 such that the injection device is ready to release the next fixed dose.

### Injection of the Next Doses

### FIG. 12B-C-D-E-F-G-H

Once the needle shield 30, 35 has been rotated to the position disclosed in FIG. 12M which is similar to the position shown in FIG. 12B and the protective cap 20 has been removed, the injection device is ready for injecting the next fixed dose using the next needle assembly 70 as will be explained.

The next dose is prepared and released by pushing the first shield 35 against the skin of the subject to be injected as described in the above state named “Preparing the First Dose” and “Injecting the First Dose”. Actually, all the following fixed doses are prepared and released in this way.

Conclusively, the helical track 102 is only used in connection with the first fixed dose and between each of the following fixed doses the pair of protrusions 33 needs to be moved to the bottom of the peripheral track 103 as disclosed in FIG. 12L in order to align the release mechanism for the next fixed dose. The injection device thus has to be locked and unlocked before a next fixed dose volume can be released.

This pattern of movements is used to also guide the needle change mechanism such that for each preparation of a new fixed dose, a new and fresh needle assembly is being brought into the injection position.

### Needle Change Mechanism

During the various states described above the needle change mechanism moves one fresh and unused needle assembly into injection position at the time as will be explained in the following.

The following table shows the correlation between the figures showing the needle change mechanism and the figures showing the different states of the operation of the injection device in regards of expelling the doses.

| Needle Change | | Injection |
|---|---|---|
| Mechanism | Action | Mechanism |
| 13A | Start Position | 12A |
| | Out-of-Package. | |
| 13B-C | First Rotation of Needle | 12A-B |
| | Shield. | |
| 13D + 14A-B | Unlocked and Ready for First | 12B |
| | Injection. | |
| 15A-B | Penetrating Needle into skin. | 12C-D |
| | Preparing First Dose | |
| 16A-B | Injection. | 12E-F-G |
| 17A-B | Needle Shield Sliding Back. | 12H |
| 18A-B | Needle Shield Fully | 12I |
| | Removed from the Skin. | |
| 19A-B | Needle Shield Being Rotated | 12J-K |
| | Towards Locked Position | |
| 20A-B | Needle Shield Fully Locked. | 12L |
| 21A-B | Needle Shield being Rotated | |
| | Towards Unlocked Position. | |
| 22 | Needle Shield in Unlocked | 12M |
| | Position | |

### First Needle

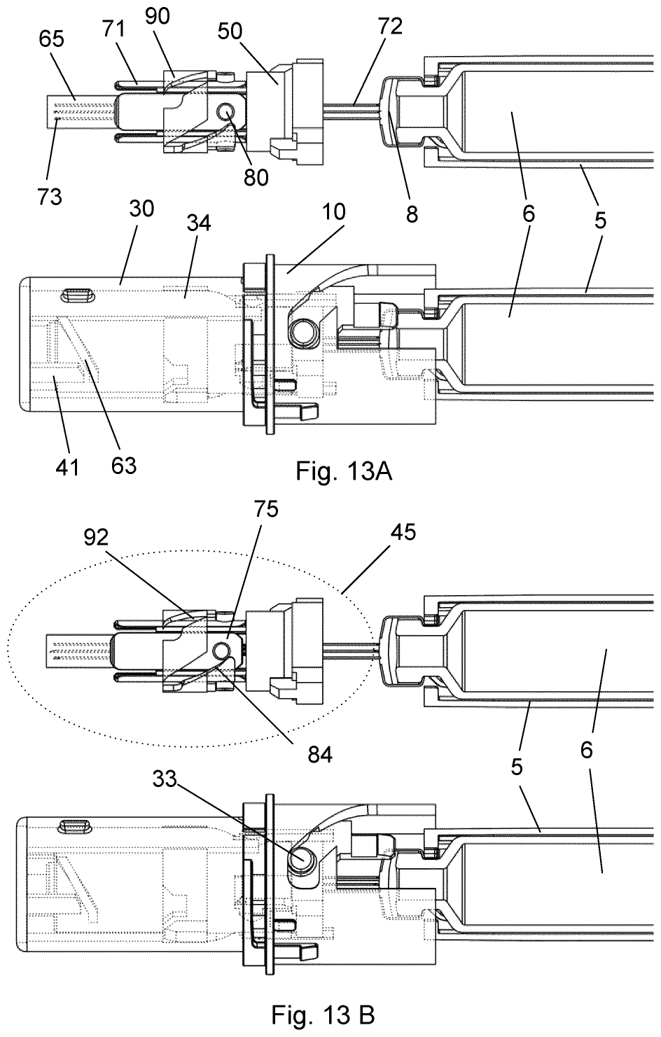

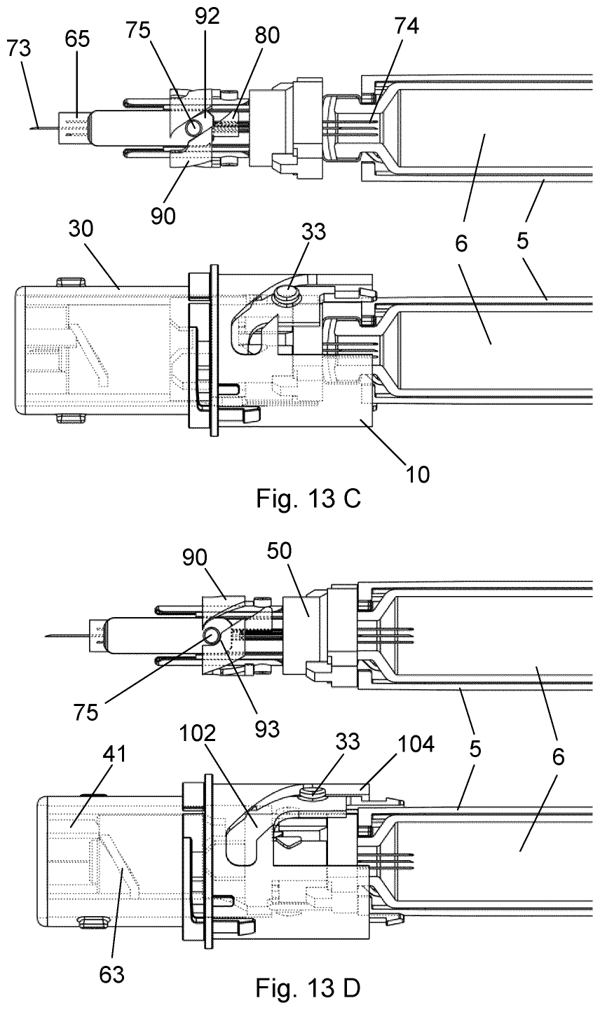

Preparing the first needle for injection is schematically disclosed in FIG. 13A-D which shows from left to right:
The needle assemblies 70 each comprising a hub 71 and a needle cannula 72.
A pierceable plug 65 in which the distal ends 73 of all the needle cannulae 72 are kept sterile.
The needle unit 45 with the tip part 60, the tower 50, the key 80 and the carousel 90.

However, the tip part 60 is only shown with broken lines as it moves one layer below the first shield 30.
The first shield 30 and the shield guide 10.
The cartridge 6 which distally is closed by the septum 8. The cartridge 6 is secured in the cartridge holder 5.

In the FIGS. 13A-D only the distal end of the injection device is disclosed. The FIGS. 13A-D each disclose an upper figure wherein the first shield 30 and the shield guide 10 has been visually removed and a lower figure wherein the needle unit 45 is shown in broken lines as an underlying structure.

Returning shortly to FIG. 5B-C, the first shield 30 is on the inner surface provided with a first shield rib 41 and a second shield rib 42. During rotation of the first shield 30, the first shield rib 41 abuts the helical rib 63 provided on the tip part 60 of the needle unit 45 as seen in figure 13A-D. This has the effect that the needle unit 45 is forced to move in the proximal direction as the needle shield 30 is rotated. During this movement, the needle unit 45 is guided to move translational.

The needle unit 45 is preferably guided translational by having a pair of radial protuberances 55 being guided in an axial track 15 provided in the shield guide 10 which is a part of the housing structure. This axial track 15 is best seen in FIG. 3A.

The rotation of the first shield 30 is also here introduced by the rotation of the protective cap 20 which engages the outwardly pointing protrusion 31 on the first shield 30. As the first shield 30 is rotated it moves helically in the proximal direction due to the guidance of the pair of protrusions 33 in the helical track 102. Further, as the first shield 30 in its helically movement also presses on the helical rib 63, the needle unit 45 is moved in the proximal direction with a high speed due to the double helical engagement (rotation of the first shield 30 drives the needle unit 45 translational in the proximal direction and rotation of the first shield 30 drives the first shield 30 helically in the proximal direction).

During the rotation of the needle shield 30 from the position disclosed in FIG. 13A to the position disclosed in FIG. 13D, the second shield rib 42 engages a flexible flap 82 provided on the key 80 such that a rotation of the needle shield 30, 35 is transformed to a similar rotation of the key 80.

The key 80 which is disclosed in FIG. 9A-D is further provided with an inner protrusion 83 which has a curved flange 84 pointing in the proximal direction. As seen in FIG. 13B wherein a part of the key 80 has been cut away to better visualize the curved flange 84, this curved flange 84 engages with the protrusion 75 provided on the needle hub 71.

During rotation of the key 80 by the first shield 30, this curved flange 84 engages the protrusion 75 on the needle hub 71. As the user continues to rotate the first shield 30, the needle unit 45 moves in the proximal direction due to engagement of the first shield rib 41 with the helical rib 63 on the needle unit 45. However, due to the engagement of the curved flange 84 with the protrusion 75, one of the needle assemblies 70 is prevented from fully following the proximal movement of the remaining needle assemblies 70 in the needle unit 45.

In fact, all the needle assemblies 70 travels with a high speed in the proximal direction except one of the needle assemblies 70 which due to the engagement between the protrusion 75 on the needle hub 71 and the curved flange 84 in the key 80 travels with a lower speed. The result being that all the proximal ends 74 of the needle cannulae 72 penetrates through the septum 8 of the cartridge 6, however, one of the needle cannulae 72 are moved a smaller distance into the cartridge 6 whereas the distal end 73 of that particular needle cannula 72 is moved to its injection position which is a position wherein the distal end 73 of the needle cannula 72 lies outside and distal to the needle unit 45.

However, the continued rotation of the first shield 30 translates the needle unit 45 including the needle assemblies 70 in the proximal direction as disclosed in FIG. 13C-D. Once the pair of protrusions 33 reaches the end of the helical track 102 and the shield rib 41 has moved the helical rib 63 to its proximally end position as shown in FIG. 13D, the proximal ends 74 of all the needle cannulae 72 has been penetrated through the septum 8 of the cartridge 6.

The rotation of the key 80 moves the protrusion 75 on the needle hub 71 into the helical guiding track 92 in the carousel 90 as disclosed in FIG. 13C. The carousel 90 follows the rotation of the first shield 35 due to the engagement between longitudinal rib 34 and the axial groove 91 in the outer surface of the carousel 90. Thus, both the key 80 and the carousel 90 follows the rotation of the first shield 35 as the pair of protrusions 33 move through the helical track 102.

During the rotation occurring from FIG. 13C to FIG. 13D, the protrusion 75 on the needle hub 71 is moved further up the helical guiding track 92 on the inner surface of the carousel 90 and at the end of the rotation, the protrusion 75 ends on a shelf 93 provided in the helical guiding track 92. In this position, the first needle assembly 70 has been moved to the injection position and the distal end 73 of the needle cannula 72 is ready to penetrate the skin of the user. Further, due to the shelf 93, the needle assembly 70 is prevented from moving in the proximal direction during injection. Also, in this position are all the proximal ends 74 of the needle cannulae 72 in liquid communication with the liquid drug inside the cartridge 6, however the specific needle cannula 72 to be used for the injection has moved a shorter distance into the cartridge 6. A distance which is similar to the distance the distal end 73 of the specific needle cannula 72 has been moved out of the plug 65.

Once the needle unit 45 has been moved translational to the position disclosed in FIG. 13D, the tower 50 of the needle unit 45 automatically locks to the cartridge holder 5 such that all the proximal ends 74 of the needle cannulae 72 hereafter is maintained irreversible inserted into the cartridge 6.

Further, in the position depicted in FIG. 13D wherein the injection device is ready to release and inject the first dose, has the first shield rib 41 passed the helical rib 63 such that the first shield rib 41 can slide freely in the proximal direction as the needle shield 30, 35 is pushed against the skin of the subject to be injected.

### Injection Using First Needle

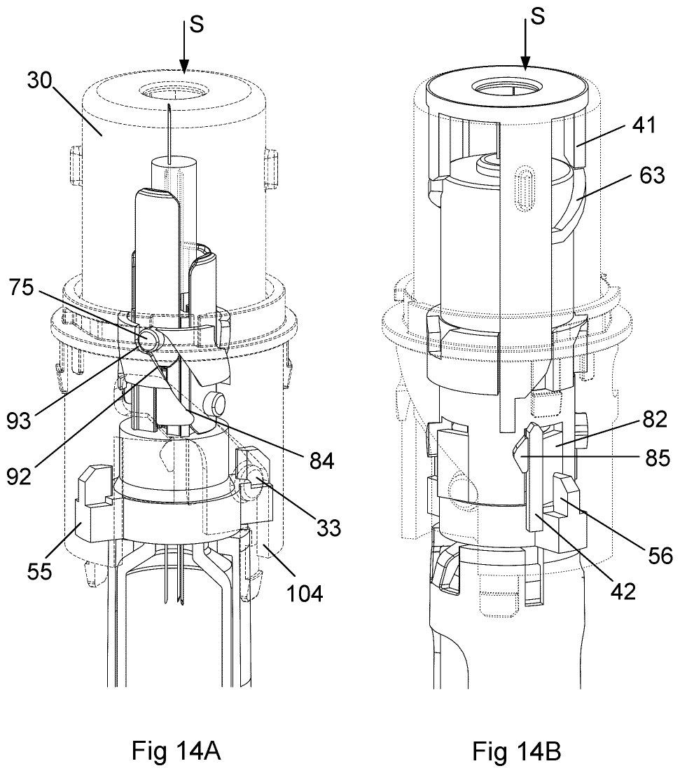

Once the first needle assembly 70 is in the injection position disclosed in FIG. 13D, the user can press the needle shield 30 against the skin of the subject to be injected. This is also disclosed in FIG. 14A-B wherein the pressure when pushed against the skin is indicated with the arrow “S”.

In the FIGS. 14A-21A (figures marked “A”), the needle shield 30 and the shield guide 10 are shown with broken lines and parts of the key 80 of the carousel 90 has been cut away to visually see the tracks on the inside of these elements. In the FIGS. 14B-21B (figures marked “B”) the outer contour of the needle shield 30 and the shield guide 10 are shown in broken lines. The FIGS. 14A-21A (figures marked “A”) are rotated approximately 90° relatively to the FIGS. 14B-21B (figures marked “B”).

Since the pair of protrusions 33 and the first shield rib 41 are free to translate in the proximal direction, the needle shield 30 will slide in the proximal direction when pushed against the skin. This proximal movement will thus compress the connector spring 143. As the first shield 30 moves proximally, the distal end 73 of the needle cannula 72 penetrates the skin of the subject being injected as the needle assembly 70 is prevented from movement in the proximal direction due to the engagement between the protrusion 75 on the needle hub 71 and the shelf 93 inside the carousel 90.

The key 80 which is disclosed in details on FIG. 9A-D, is provided with a number of flexible flaps 82 and an identical number of radial protrusions 85 on the outer surface. Each of the radial protrusions 85 has two sloped surface to facilitate rotation of the key 80 as will be explained.

In FIG. 14B, at least one of these radial protrusions 85 abut the second shield rib 42 which also abuts the protuberance 55 such that the key 80 is inrotatable maintained in its position.

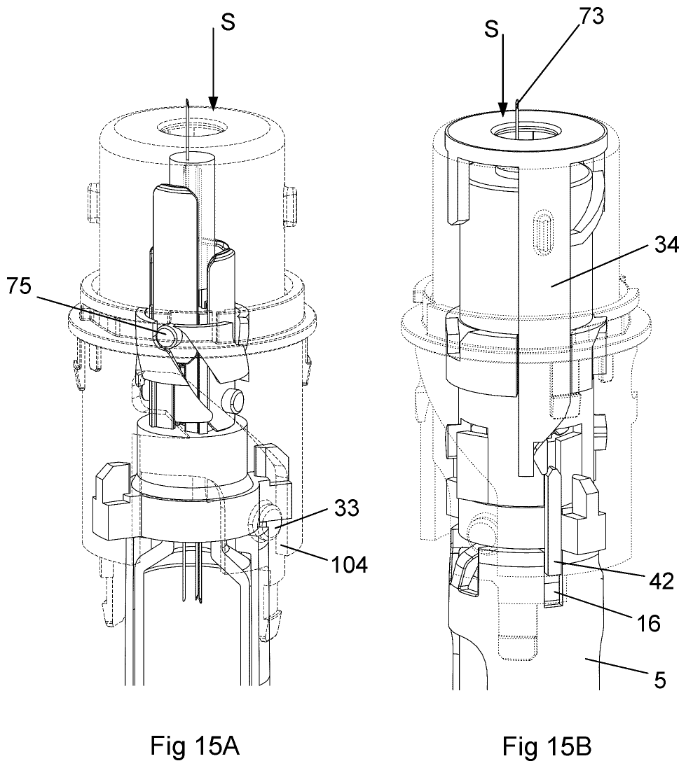

As the needle shield 30 is moved further in the proximal direction as disclosed in FIG. 15A-B, the distal end 73 of the needle cannula 72 penetrates deeper into the skin of the subject being injected and the pair of protrusions 33 moves translational in the proximal direction in the axial track 104. At the same time the second shield rib 42 which is guided in longitudinal tracks 16 in the cartridge holder 5 and the longitudinal rib 34 on the inner surface of the first shield 30 moves in the proximal direction.

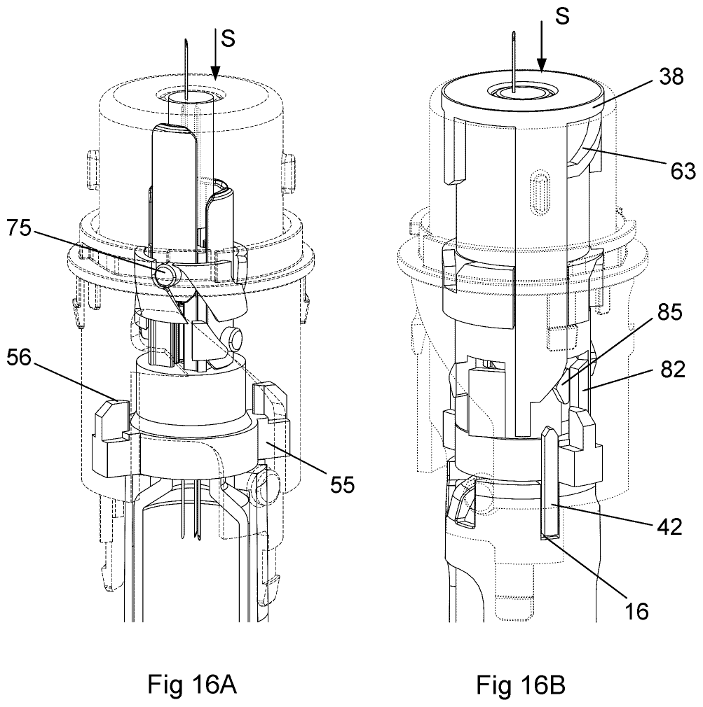

The longitudinal rib 34 is at the proximal end provided with an axial extension 43 and a sloped surface 44 which as the needle shield 30 is moved proximally engages with the radial protrusion 85 on the key 80 and as disclosed in FIG. 16A-B starts to rotate the radial protrusion 85 and the key 80 in the counter clock-wise direction once the radial protrusion 85 is no longer abutting the second shield rib 42.

FIG. 16A-B discloses the situation in which the distal end 73 of needle cannula 72 is fully inserted into the skin of the subject being injected. In this position the inner part of the front end 38 of the needle shield 30 engages with the helical rib 63 on the needle unit 45 which prevents the needle shield 30 from moving further in the proximal direction. This is further prevented due to the engagement between the second shield rib 42 and the bottom of the longitudinal tracks 16.

The protuberance 55 on the tower 50 of the needle unit 45 is distally provided with a blade 56. This blade 56 allows the flexible flap 82 to pass by the blade 56 in one direction (counter clockwise in this example) but prevents the flexible flap 82 from passing the blade 56 in the opposite direction (clock-wise).

In FIG. 16A-B the rotation of the key 80 introduced by the translation of the needle shield 30 has forced the flexible flap 82 to pass by the blade 56 and are thus in a position in which the key 80 is prevented from rotating back in the clock-wise direction. The rotation of the key 80 also rotates the curved flange 84 on the key 80 towards the next needle assembly 70 as seen in FIG. 16A.

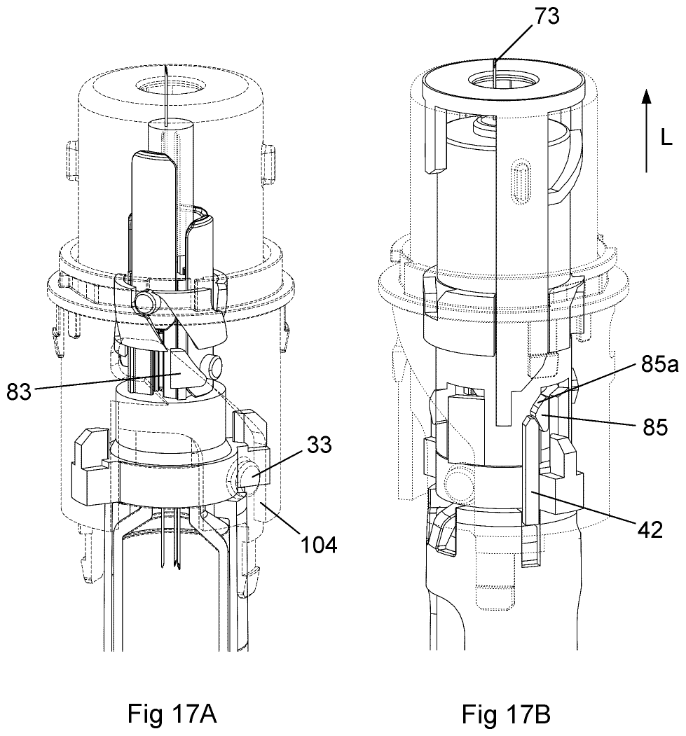

Once the fixed dose volume has been delivered, the user removes the needle shield 30 from the skin of the subject being injected. The connector spring 143 will consequently move the first shield 30 in the distal direction as disclosed in FIG. 17A-B. This translational movement is indicated by the arrow “L” in FIG. 17B. During this translation of the needle shield 30, the second shield rib 42 abut the radial protrusion 85. The impact with the second shield rib 42 will rotate the radial protrusion 85 and thus the key 80 further in the counter clock-wise direction due to the second sloped surface of the radial protrusion 85.

As seen e.g. in FIG. 9B, the radial protrusion 85 has one sloped surface (85a) which the sloped surface 44 on the longitudinal rib 34 engages when the needle shield 30 moves in the proximal direction and a second sloped surface (85b) which is impacted by the second shield rib 42 when the needle shield 30 moves in the distal direction as shown in FIG. 17A-B.

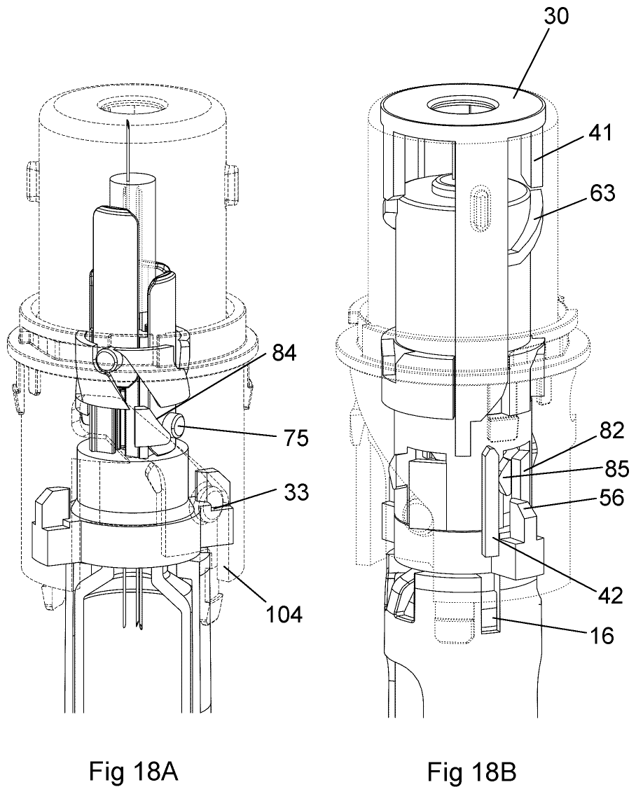

FIG. 18A-B discloses the situation wherein the needle cannula 72 has been fully removed from the skin and is again covered by the needle shield 30. In this position, the pair of protrusions 33 has moved to the distal end of the axial track 104 and is positioned as disclosed in FIG. 12L. The key 80 has been rotated to a position in which the curved flange 84 is located adjacent the protrusion 75 on the next needle assembly 70.

### Retraction of First Needle

As seen in FIG. 12J and 12K, the user rotates the needle shield 30 once the injection has been preformed and the needle shield 30 has been removed from the skin. This is indicated by the arrow “C” and creates a rotational movement of the pair of protrusions 33 in the peripheral track 104. The clock-wise rotation of the needle shield 30 is generated when the user attaches the protective cap 20 to the shield guide 10 using the peripheral track 12.

As explained earlier and shown on FIG. 10A-D, the carousel 90 is provided with a number of grooves 91 which engages with the longitudinal ribs 34 provided inside the needle shield 30 such that the carousel 90 rotates together with the needle shield 30 at all times.

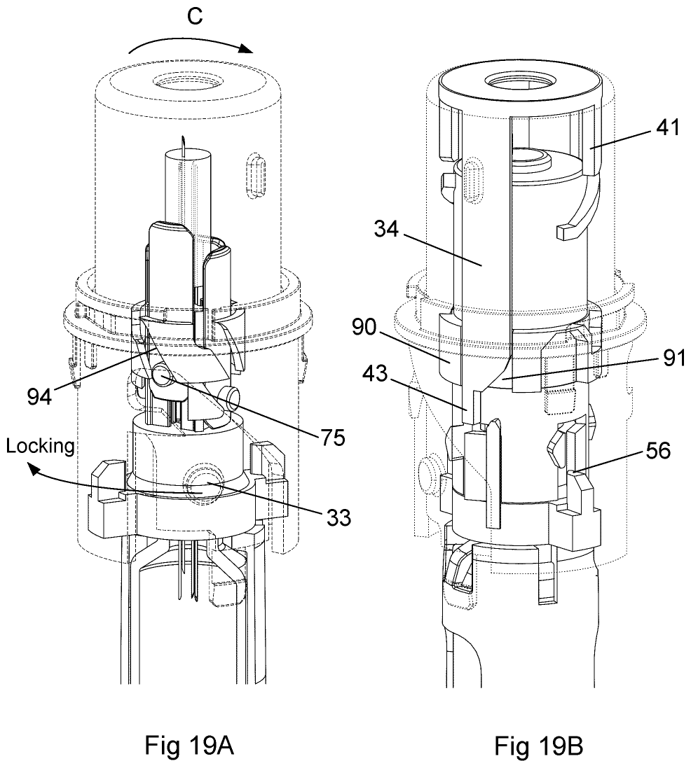

As best seen in FIG. 19A-B, the rotation of the needle shield 30 simultaneously rotates the longitudinal rib 34 and the second shield rib 42 as theses ribs (34, 41, 42) are integral parts of the needle shield 30. This rotation occurs in the clock-wise direction as indicated by the arrow “C” in FIG. 19A as the pair protrusions 33 rotates in the peripheral track 103 as also indicated in FIG. 19A. The rotation is also transformed to a rotation of the carousel 90. However, the key 80 is prevented form rotation in the clock-wise direction by the engagement between the flexible flap 82 and the blade 56 provided on the needle unit 45 as best seen in FIG. 19B.

As the carousel 90 rotates together with the needle shield 30, the sidewall 94 of the helical track (see FIG. 100) will abut the protrusion 75 on the now used needle assembly 70 and force the needle assembly 70 to move in the proximal direction. This axial movement will be a translation due to the engagement between rib configuration 76 on the needle assembly 70 and the longitudinal rack structure 53 on the tower 50 of the needle unit 45.

Once the protective cap 20 has been fully attached and the inner protrusion 21 inside the protective cap 20 has been moved to its locked position which is indicted by the arrow “21” in FIG. 12L, the pair of protrusions 33 has been moved to the end of the peripheral track 103 as also disclosed in FIG. 12L.

This situation is reflected in FIG. 20A-B which also disclose that in this position has the second shield rib 42 passed the flexible flap 82 on the key 80 in the clock-wise direction and the protrusion 75 on the used needle assembly 70 has been moved down and into the key 80.

As best seen in FIG. 9D, the key 80 is provided with an inwardly pointing radial flange 86 at its distal end. This radial flange 86 is separated from the protrusion 83 by axial openings 87a-b which are located adjacent to the protrusion 83 on both rotational sides.

These openings 87a-b allows the protrusion 75 on the needle assembly 70 to move from the helical track 92 in the carousel 90 and back into key 80. Once the protrusion 75 is moved back into the carousel 90, the needle assembly 70 is fully retracted. This is e.g. seen in FIG. 20A wherein all the needle assemblies 70 are now in the same axial position.

In this stored position of the needle assemblies 70 are all the proximal ends 74 of the needle cannulae located inside the cartridge 6 and the distal ends 73 are all stored in the plug 65 which preferably contains an anti bacterial material.

Moving Next Needle into Injection Position

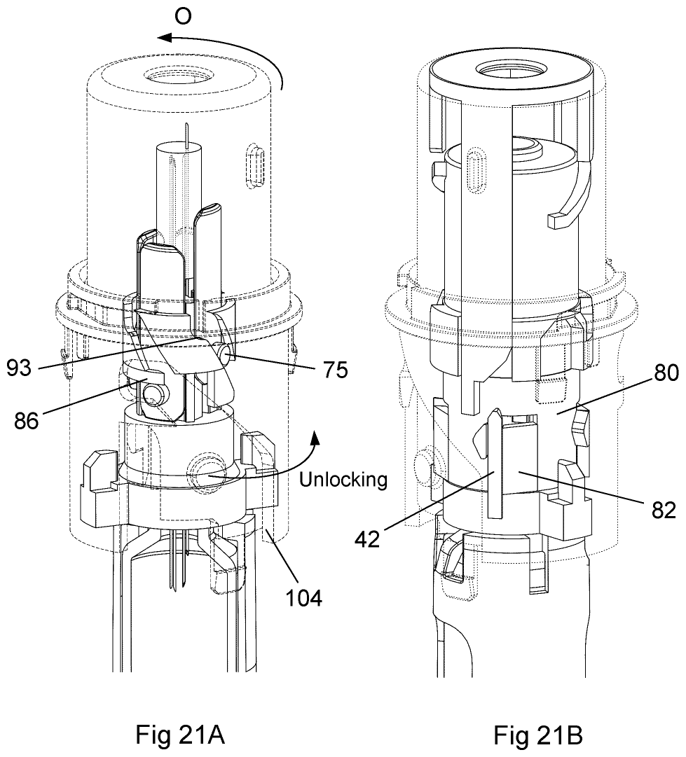

The injection device is hereafter unlocked by rotating the needle shield 30 in the counter clock-wise direction as indicated by the arrow “O” in FIG. 21A which is automatically done when the user rotates the protective cap 20 to remove it.

During this rotation which is disclosed in the transition from FIG. 12L to FIG. 12M, the pair of protrusions 33 moves through the peripheral track 103 from the position in FIG. 12L to the position in FIG. 12M.

It is noted that since the peripheral track 103 is a part of the housing part 100 it is not physically shown in the FIGS. 13A to 22. However, the position of the pair of protrusions 33 are disclosed in these figures. The direction in which the pair of protrusions 33 rotates when locking and unlocking the injection device is disclosed in FIG. 19A (locking) and in FIG. 21A (unlocking).

When the user rotates the needle shield 30 in the counter clock-wise direction to unlock the injection device, the second shield rib 42 engages the flexible flap 82 and rotates the key 80 also in the counter clock-wise direction. This rotation lifts the protrusion 75 on the next needle assembly 70 in the distal direction as the curved flange 84 on the key 80 engages the protrusion 70 on the needle hub 71.

The radial flange 86 on the key 80 prevents the other needle assemblies 70 in moving axially as the radial flange 86 abut the protrusions 75 as seen in FIG. 21A.

The needle shield 30 also engages and rotates the carousel 90 such that the carousel rotates together with the needle shield 30 and with the key 80. This has the consequence that the protrusion 75 is first picked up by the curved flange 84 inside the key 80 and delivered into the helical track 91 of the carousel 90 and the next needle assembly 70 is thus lifted to the shelf 93. As seen in FIG. 21A-B, as the pair of protrusions 33 approaches the axial track 104, the protrusion 75 on the next needle assembly 70 is being moved distally towards the shelf 93 and the distal end 73 of the needle canula 72 moves out of the plug 65 and toward the injection position.

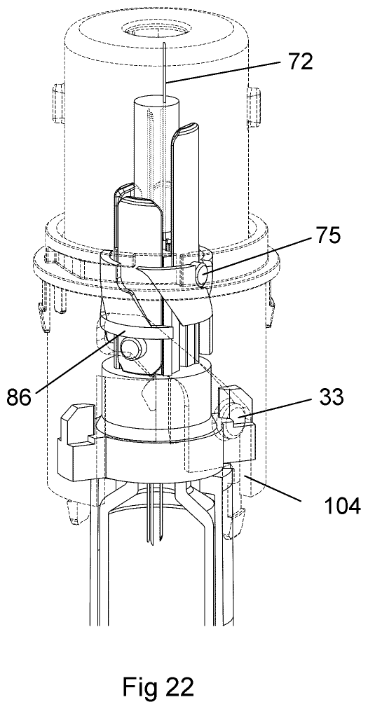

The injection position is the position wherein the protrusion 75 on the individual needle hub 71 is located on the relevant shelf 93 and the distal end 73 of the needle cannula 72 has been moved out of the plug 65. This position is disclosed in FIG. 22.

In this position which is the same position as disclosed in FIG. 14A-B and in FIG. 12B and 12M, the injection device is ready to perform the next injection using the next needle assembly 70.

A comparison between FIG. 14A and FIG. 22 shows that the first needle assembly 70 has been moved proximally, and a new fresh needle assembly 70 has been moved to the injection position (the shelf 93). At the same time, the injection device has been prepared to a new injection as the inwardly pointing protrusion 142 on the connector element 140 has been moved to a position abutting the axial rib 131 on the drive tube 130 as seen in FIG. 12B and in FIG. 12M.

### Summary of the Injection Mechanism and the Needle Change Mechanism

The injection device is thus delivered to the user with the needle unit 45 decoupled from the housing structure as disclosed in FIG. 13A. In this position, the proximal ends of the needle cannulae 74 are all preferably covered by a not-shown bag-like construction such that the proximal ends 74 can be maintained sterile when the injection device is stored.

Then, first time the user removes the protective cap 20, the needle shield 30 is rotated and moved helically due to the engagement of the pair of protrusion 33 with the helical track 102 in the housing structure. This first rotation of the needle shield 30 translates the needle unit 45 in the proximal direction such that all the proximal ends 74 of the needle cannulae 72 penetrates the septum 8 of the cartridge 6 and thus gains liquid contact with the drug inside the cartridge 6. In the remaining lifetime of the injection device all the proximal ends 74 are maintained inserted into the cartridge 6.

Simultaneously with the insertion of the needle cannulae 72 into the cartridge 6, the needle change mechanism lifts the first needle assembly 70 into the injection position as disclosed in FIG. 13D and FIG. 14A-B. This position is also disclosed in FIG. 12B.

During injection, the needle shield 30 translates proximally which starts to rotate the key 80 towards the next needle assembly 70 as disclosed in FIG. 15A-B and in FIG. 16A-B. Since the needle shield 30 does not rotate during injection, the carousel 90 remains in its rotational position.

Once the injection has been performed and the needle shield 30 is slided back to its extended position as disclosed in the FIGS. 17A-B, the key 80 is rotated further in the counter clock-wise direction such that the curved flange 84 abuts the next needle assembly. This situation is also reflected in FIG. 12L.

In order to re-align the dosing mechanism, the user needs to rotate the needle shield 30 to its locked position and back to its unlocked position. If this is not done, a new dose cannot be released. The rotation of the needle shield 30 is, as explained, facilitated by the attachment and the removal of the protective cap 20.

The locking of the injection device is disclosed in FIG. 19A-B. Here the user rotates the needle shield 30 in the clock-wise direction. During this rotation of the needle shield 30, the carousel 90 is rotated together with the needle shield 30 whereas the key 80 is maintained in its position.

The key 80 was thus rotated to the next needle assembly 70 during translation forth and back of the needle shield 30 as done during injection and the carousel 90 is rotated during rotation of the needle shield 30.

When the needle shield 30 is rotated to its locked position, the old and used needle assembly 70 is moved proximally back into the storage position and when the needle shield 30 is rotated to its unlocked position, the key 80 is already placed at the next needle assembly 70 which is thus lifted to the injection position as the key 80 and the carousel 90 are rotated simultaneously together in the counter clock-wise direction during the unlocking of the injection device.

The change of the needle assemblies 70 are thus done by translating the needle shield 30 forth and back followed by a locking and an unlocking of the injection device. This can in theory be done any number of times but in the shown example a proximal stop surface is provided in the track structure 3 in the piston rod 2 which determines how many times the drive tube 130 can be moved proximally which again determines how many fixed doses it is possible to release. This End-of-Content mechanism is further disclosed in International patent application No. PCT/EP2000/085271 and is in the preferred example set such that the number of fixed doses to be released is the same as the number of needle assemblies 70 which in the disclosed example is four (4).

It is thus, generally preferred for the described invention that the predetermined number of doses available is the same as the number of needle assemblies available such that each predetermine dose volume can be injected through a new and clean needle cannula and such that the pre-filled injection device can be discarded once the number of fixed doses and the number of needle assemblies has been used. Should the number of fixed doses and needle assemblies be different it is important that there are more needle assemblies than fixed doses thus maintaining the basic idea of having a new and clean needle assembly available for each fixed dose. In such example, the user must then discard an injection device with one or more unused needle assemblies.

Alternatively, the key 80 can be provided with an extra protrusion which engages an inner peripheral track in the housing structure such that the key 80 can only be rotated a little less than one full revolution in relation to the housing structure. This will assure that all the needle assemblies 70 can only be used once.

Some preferred examples have been shown in the foregoing, but it should be stressed that the invention is not limited to these, but may be embodied in other ways within the subject matter defined in the following claims. It is especially pointed out that all though the number of fixed doses and the number of needle assemblies are the same, the number of these can be any relevant number and is not limited to four (4) as in the disclose example.

1. A pre-filled injection device for ejecting a predefined plurality of fixed doses of a liquid drug, comprising:
a housing structure with a container containing the predefined number of fixed doses of liquid drug to be ejected,
a release mechanism, for releasing the predefined plurality of fixed doses one at the time as a first dose followed by a number of next doses,
a plurality of needle assemblies integrated in the housing structure and individually movable into an injection position,
a needle change mechanism which when activated moves one needle assembly into the injection position,
wherein the release mechanism and the needle change mechanism cooperates such that each of the next doses can only be released after at least one activation of the needle change mechanism.

2. The pre-filled injection device according to claim 1, wherein a needle shield is telescopically movable in relation to the housing structure between a first position to a second position.

3. The pre-filled injection device according to claim 2, wherein each of the predefined equally sized dose volumes are individually released upon axial movement of the needle shield from the first position to the second position.

4. The pre-filled injection device according to claim 1, wherein the release mechanism comprises a connector element and a torsion spring (115) driven drive tube for driving a piston rod.

5. The pre-filled injection device according to claim 2, wherein the needle shield is rotatable between a locked and an unlocked position in the first position.

6. The pre-filled injection device according to claim 1, wherein, each needle assembly comprising a hub adapted to hold a needle cannula such that a distal end points in a distal direction and a proximal end points in a proximal direction and/or wherein the container is a cartridge having an interior containing the liquid drug and being defined by a movable plunger and pierceable septum,

7. The pre-filled injection device according to claim 6, wherein the proximal end of all the needle cannulae in the plurality of needle assemblies are pierced through the septum of the container upon first rotation of the needle shield from the locked position to the unlocked position.

8. The pre-filled injection device according to claim 2, wherein both the release mechanism and the needle change mechanism are activated by movement of the needle shield.

9. The pre-filled injection device according to claim 8, wherein one needle assembly is moved to the injection position upon rotation of the needle shield.

10. The pre-filled injection device according to claim 8, wherein the needle change mechanism comprises a key and a carousel.

11. The pre-filled injection device according to claim 10, wherein the translation of the needle shield generates a rotation the key.

12. The pre-filled injection device according to claim 10, wherein rotation of the needle shield generates a simultaneously rotation of the carousel.

13. The pre-filled injection device according to claim 10, wherein simultaneously rotation of the carousel and the key in the same rotational direction moves one needle assembly to the injection position.

14. The pre-filled injection device according to claim 10, wherein rotation of the carousel in the opposite rotational direction moves one needle assembly in the proximal direction and away from the injection position.

15. The pre-filled injection device according to claim 14, wherein the key is provided with structure for maintaining the key in its position during rotation of the needle shield in the opposite rotational direction.
